(19) United States
(12) Patent Application Publication
(10) Pub. No.: US 2024/0342281 A1
(43) Pub. Date: Oct. 17, 2024

(54) USE OF A STROMAL ANTIGEN TO DELIVER CELL-BASED CANCER THERAPY TO A SOLID TUMOR

(71) Applicant: The Regents of the University of California, Oakland, CA (US)

(72) Inventors: Wendell A. LIM, San Francisco, CA (US); Gregory ALLEN, San Francisco, CA (US)

(21) Appl. No.: 18/580,141

(22) PCT Filed: Aug. 3, 2022
(86) PCT No.: PCT/US2022/074489

§ 371 Date: Jan. 17, 2024

Related U.S. Application Data
(60) Provisional application No. 63/286,985, filed on Dec. 7, 2021.
(60) Provisional application No. 63/231,186, filed on Aug. 9, 2021.

(51) Int. Cl. A61K 39/00; A61P 35/00; C12N 5/0783

(52) U.S. Cl. CPC A61K 39/4631; A61K 39/4632; A61K 39/464402; A61K 39/464408; A61K 39/464458; A61K 39/464466; A61K 39/46447; A61P 35/00; C12N 5/0636; C12N 2510/00

(57) ABSTRACT

Provided herein is a cytotoxic immune cell that is primed by and/or whose cytotoxicity within the tumor microenvironment is enhanced by binding to a stromal marker, e.g., Fibroblast Activation Protein Alpha (FAP). In some embodiments, the cells may contain a protein circuit that contains at least two components, wherein one of the components binding-triggered transcriptional switch that is activated by binding to the stromal marker. The second component may be a nucleic acid encoding an immune receptor (e.g., a chimeric antigen receptor or TCR) that is activated by binding to a cancer-specific antigen and/or a pro-inflammatory cytokine.

### CROSS-REFERENCING

This application claims the benefit of U.S. provisional application Ser. No. 63/231,186, filed on Aug. 9, 2021, and 63/286,985, filed on Dec. 7, 2021, which applications are incorporated by reference herein for all purposes.

### STATEMENT REGARDING FEDERALLY SPONSORED RESEARCH

This invention was made with government support under grant nos. R01 CA196277, R01 CA249018, and U54 CA244438 awarded by The National Institutes of Health. The government has certain rights in the invention.

### INCORPORATION BY REFERENCE OF SEQUENCE LISTING PROVIDED AS AN XML FILE

A Sequence Listing is provided herewith as a Sequence Listing XML, “UCSF-647WO_Seq_Listing” created on Aug. 2, 2022 and having a size of 37 KB. The contents of the Sequence Listing XML are incorporated by reference herein in their entirety.

### INTRODUCTION

Despite the remarkable success of anti-CD19 Chimeric Antigen Receptor (CAR) T cells in treating B cell cancers the application of CAR T cells to solid tumors has proven far more difficult. There are two significant challenges in developing CAR T cells for solid tumors. The first challenge is identifying truly tumor-specific cell surface antigens to target. The second challenge is to overcome the immunosuppressive microenvironment found in solid tumors.

With respect to the first challenge, there do not appear to be ideal single antigen targets: attacking most tumor associated antigens leads to severe adverse effects associated with cross-reaction with normal organs that also express the antigen. Even the FDA approved CD19 CAR T cells have “on-target/off-tumor” killing—in this case the destruction of normal B cells. While the B cell compartment is expendable, such toxicity would be unacceptable for CAR T cells that cross-react with epithelial organs that are indispensable and cannot be eliminated.

Current strategies for targeting solid tumors primarily focus on tumor-associated antigens which are over-expressed in tumors compared to normal tissue—for example HER2 (ERBB2). Unfortunately, attempts to target tumor-associated antigens have been plagued by “on-target/off-tumor” toxicities due CAR T cells acting against normal tissue that express tumor-associated antigens at a low level. In the case of HER2 “on-target/off-tumor” toxicity led to unexpected and fatal pulmonary edema. Follow up HER2 targeted CAR T cell trials with a lower potency CAR T cell product (decreased dose, scFv affinity) have had limited efficacy. Similar dose limiting “on-target/off-tumor” toxicities have also been seen in clinical trials of CAR T cells targeting CEACAM5, EGFR, Mesothelin and CA9 due to pulmonary, intestinal or biliary tract toxicity. In addition, several other promising CAR T cell products have had to be abandoned or weakened in pre-clinical development due to excessive “on-target/off-tumor” toxicity in murine models, including those targeting the antigens SSEA4, B7-H4, GD2, and FAP.

With respect to the second challenge, it is well established that immunosuppressive cells are recruited into the tumor microenvironment. For example, solid tumors often contain a variety of different immunosuppressive cells, including a specialized subset of CD4+ T cells called regulatory T cells or Tregs. Tregs are highly immunosuppressive and play a crucial role in maintaining immune tolerance during homeostasis and suppressing exacerbated immune responses in various pathological conditions. These cells have been shown to suppress the anti-tumor immune response and promote tumor growth. As such, solid tumors often suppress the desired effects of cytotoxic T cells that target such tumors.

Many current efforts in T cell engineering are focused on trying to increase the potency and durability of CAR T cells to overcome suppressive solid tumor microenvironments. However, if such increases in CAR T potency are not coupled with an improved ability to kill only tumor cells, then the adverse effects caused by off-tumor cross-reactivities may become even more severe and lethal.

Thus, there is a great need for next generation cellular therapies to more precisely distinguish tumor tissue from normal tissue and overcome the tumor microenvironment, for the treatment of solid tumors.

### SUMMARY

The present disclosure provides a cytotoxic immune cell that is primed by and/or whose cytotoxicity within the tumor microenvironment is enhanced by binding to a stromal marker, e.g., FAP (Fibroblast Activation Protein Alpha), (ii) PDPN (Podoplanin), (iii) CDH11 (Cadherin 11), (iv) PDGFR (Platelet-derived growth factor) or (v) LRRC15 (Leucine Rich Repeat Containing protein 15). FAP and other stromal markers can be overexpressed by fibroblasts in the microenvironment of many fibrotic tumors, including pancreatic ductal adenocarcinoma (PDAC), stomach adenocarcinoma, lung adenocarcinoma, mesothelioma, colon adenocarcinoma and sarcoma, among others. As such, the present cells can be used for the treatment of a variety of different cancers. In some embodiments, the cells may contain a circuit that contains at least two components, wherein one of the components is a binding-triggered transcriptional switch that is activated by binding to the stromal marker. The second component may be a nucleic acid encoding (a) a chimeric antigen receptor that is activated by binding to a cancer-specific antigen such as an antigen listed in Table 1 (depending on the type of tumor that is being target by the cell), or (b) a pro-inflammatory protein (which term refers to natural molecules such as IL-2, CCL-21, IL-12, IL-7, IL-15 and IL-21, as well as non-natural molecules that have pro-inflammatory activity such as super IL2, mini-TGF-Beta (which blocks TGF-Beta signaling) and DR-18 (an IL-18 variant), etc.). In some embodiments, the transcription factor released from the BTTS may activate expression of the second component. In some embodiments, the second component may be one or more nucleic acids encoding a chimeric antigen receptor and a pro-inflammatory protein. In these embodiments, the transcription factor released from the BTTS may activate expression of both the chimeric antigen receptor and a pro-inflammatory protein.

A tumor ecosystem is made up of multiple cells, including cancer cells and cancer associated stromal cells (e.g., cancer associated fibroblasts). In some embodiments described in this disclosure, therapeutic T cells contain two-stage circuit (i.e., a “prime-and-kill” BTTS to immune receptor circuit) that integrates information from two antigens, each expressed on a different cell type (cancer cell vs stromal cell). These “prime-and-kill” T cells first receive a “priming antigen” signal (from tumor stromal cells), which then licenses the T cell to be able to kill the neighboring cancer cells based on a second “killing antigen” (by inducing expression of a CAR for the killing antigen). In this case, the target cancer cells must be within the same tissue, or within the “killing radius” of the priming signals (the effective radius around priming cells that primed T cells can survey). The therapeutic cells obtain a “short-term memory” of priming, which allows them to kill neighboring cancer cells, for the duration of this memory. Because this is a short, local memory (immune receptor CAR expression decays in hours), T cells are prevented from mounting a strong attack on distant normal tissues that share the “killing antigen” but lack the “priming antigen.” This circuit design is believed to be innovative and distinct because no other combinatorial antigen recognition scheme is able to combinatorially recognize antigens presented on distinct cell types.

Alternatively or in addition to containing a “prime-and-kill” BTTS to immune receptor circuit, a cell may also contain a BTTS to pro-inflammatory protein circuit. In these embodiments, expression of the pro-inflammatory protein helps overcome the immunosuppressive environment that typically exists in the tumor microenvironment, thereby enhancing the ability of the cells to kill cancer cells.

When both the immune receptor and the protein are both induced by binding of the BTTS to the stromal marker, the cytotoxicity of those cells in the tumor microenvironment (as well as other immune cells that are local to the primed cells) should be increased, while, at the same time, cross-reactivity with normal tissue is avoided. Such therapeutic cells therefore meet a great need.

This kind of integrative therapeutic T cell would provide a generalizable strategy for treating many types of solid cancers. Cancer associated fibroblasts are found in many solid tumors and could be combined with different disease-specific killing antigen targets to target a broad array of cancers.

Examples of such circuits and their use are described in further detail below.

### BRIEF DESCRIPTION OF THE DRAWINGS

FIG. 1: Validation of anti-FAP-synNotch. (a) A synNotch receptor designed to recognize FAP was connected to a transcriptional response element designed to produce BFP in primary human CD8+ T cells. (b) Co-culture of this circuit with FAP expressing cells (pancreatic stellate cells) led to induction of BFP production in the engineered CD8+ T cells.

FIG. 2: anti-FAP synNotch to anti-MSLN CAR circuit T cells clear PDAC xenograft tumors with reduced toxicity: (a) A synNotch receptor designed to recognize FAP was connected to a transcriptional response element designed to produce an anti-mesothelin CAR T cell. (b) Primary human CD8+ T cells expressing this transcriptional circuit cleared cultures of PANC04 PDAC cells only when co-cultured with FAP+stellate cells, which acted as priming cells. (c) This transcriptional circuit controlled the growth of human PDAC (PANC04) xenograft tumors in NSG mice with similar efficacy to a standard anti-mesothelin CAR T cell. (d) results of an in vivo T cell toxicity assay.

FIG. 3: Constitutive anti-MSLN-CAR plus an anti-FAP synNotch to IL2 circuit clears PDAC syngeneic tumor model (KPC) (a) A synNotch receptor designed to recognize FAP was connected to a transcriptional response element designed to produce an anti-mesothelin CAR T cell and IL-2. (b) Primary mouse CD3+ T cells expressing this transcriptional circuit had superior tumor control when compared to standard anti-mesothelin CAR T cells in the KPC tumor model in C57/B16 background. (c) This transcriptional circuit led to improved overall survival in the KPC tumor model in the C57/B16 background.

FIG. 4: Other tumor types that could be targeted by FAP induced priming (anti-FAP synNotch) Many potential combinatorial detection strategies for other cancers including (Lung Adenocarcinoma, Stomach Adenocarinoma, Mesothelioma, Sarcoma and Colon Adenocarcinoma) could involve priming off of the tumor stromal antigen FAP.

FIG. 5: Non small cell lung cancer can be treated using CAR T cells that have a FAP-EGFR circuit. This data shows that CART T cells that are engineered to contain a FAP activatable BTTS and an EGFR activatable CAR can be used to treat EGFR+ non-small cell lung cancer and other solid tumors that are EGFR+.

### DEFINITIONS

As used herein, the terms “treatment,” “treating,” “treat” and the like, refer to obtaining a desired pharmacologic and/or physiologic effect and/or a response related to the treatment. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which can be predisposed to the disease but has not yet been diagnosed as having it; (b) inhibiting the disease, i.e., arresting its development; and (c) relieving the disease, i.e., causing regression of the disease.

A “therapeutically effective amount” or “efficacious amount” refers to the amount of an agent (including biologic agents, such as cells), or combined amounts of two agents, that, when administered to a mammal or other subject for treating a disease, is sufficient to effect such treatment for the disease. The “therapeutically effective amount” will vary depending on the agent(s), the disease and its severity and the age, weight, etc., of the subject to be treated.

The terms “individual,” “subject,” “host,” and “patient,” used interchangeably herein, refer to a mammal, including, but not limited to, murines (e.g., rats, mice), non-human primates, humans, canines, felines, ungulates (e.g., equines, bovines, ovines, porcines, caprines), lagomorphs, etc. In some cases, the individual is a human. In some cases, the individual is a non-human primate. In some cases, the individual is a rodent, e.g., a rat or a mouse. In some cases, the individual is a lagomorph, e.g., a rabbit.

The term “refractory”, used herein, refers to a disease or condition that does not respond to treatment. With regard to cancer, “refractory cancer”, as used herein, refers to cancer that does not respond to treatment. A refractory cancer may be resistant at the beginning of treatment or it may become resistant during treatment. Refractory cancer may also called resistant cancer.

The term “histology” and “histological” as used herein generally refers to microscopic analysis of the cellular anatomy and/or morphology of cells obtained from a multicellular organism including but not limited to plants and animals.

The term “cytology” and “cytological” as used herein generally refers to a subclass of histology that includes the microscopic analysis of individual cells, dissociated cells, loose cells, clusters of cells, etc. Cells of a cytological sample may be cells in or obtained from one or more bodily fluids or cells obtained from a tissue that have been dissociated into a liquid cellular sample.

The terms “chimeric antigen receptor” and “CAR”, used interchangeably herein, refer to artificial multi-module molecules capable of triggering or inhibiting the activation of an immune cell which generally but not exclusively comprise an extracellular domain (e.g., a ligand/antigen binding domain), a transmembrane domain and one or more intracellular signaling domains. The term CAR is not limited specifically to CAR molecules but also includes CAR variants. CAR variants include split CARs wherein the extracellular portion (e.g., the ligand binding portion) and the intracellular portion (e.g., the intracellular signaling portion) of a CAR are present on two separate molecules. CAR variants also include ON-switch CARs which are conditionally activatable CARs, e.g., comprising a split CAR wherein conditional hetero-dimerization of the two portions of the split CAR is pharmacologically controlled (e.g., as described in PCT publication no. WO 2014/127261 A1 and US Patent Application No. 2015/0368342 A1, the disclosures of which are incorporated herein by reference in their entirety). CAR variants also include bispecific CARs, which include a secondary CAR binding domain that can either amplify or inhibit the activity of a primary CAR. CAR variants also include inhibitory chimeric antigen receptors (iCARs) which may, e.g., be used as a component of a bispecific CAR system, where binding of a secondary CAR binding domain results in inhibition of primary CAR activation. CAR molecules and derivatives thereof (i.e., CAR variants) are described, e.g., in PCT Application No. US2014/016527; Fedorov et al. Sci Transl Med (2013);5(215):215ra172; Glienke et al. Front Pharmacol (2015) 6:21; Kakarla & Gottschalk 52 Cancer J (2014) 20(2):151-5; Riddell et al. Cancer J (2014) 20(2):141-4; Pegram et al. Cancer J (2014) 20(2):127-33; Cheadle et al. Immunol Rev (2014) 257(1):91-106; Barrett et al. Annu Rev Med (2014) 65:333-47; Sadelain et al. Cancer Discov (2013) 3(4):388-98; Cartellieri et al., J Biomed Biotechnol (2010) 956304; the disclosures of which are incorporated herein by reference in their entirety. Useful CARs also include the anti-CD19-4-1BB-CD3(CAR expressed by lentivirus loaded CTLO19 (Tisagenlecleucel-T) CAR-T cells as commercialized by Novartis (Basel, Switzerland).

The terms “T cell receptor” and “TCR” are used interchangeably and will generally refer to a molecule found on the surface of T cells, or T lymphocytes, that is responsible for recognizing fragments of antigen as peptides bound to major histocompatibility complex (MHC) molecules. The TCR complex is a disulfide-linked membrane-anchored heterodimeric protein normally consisting of the highly variable alpha (a) and beta (0) chains expressed as part of a complex with CD3 chain molecules. Many native TCRs exist in heterodimeric ap or T6 forms. The complete endogenous TCR complex in heterodimeric ap form includes eight chains, namely an alpha chain (referred to herein as TCRα or TCR alpha), beta chain (referred to herein as TCRβ or TCR beta), delta chain, gamma chain, two epsilon chains and two zeta chains. In some instance, a TCR is generally referred to by reference to only the TCRα and TCRβ chains, however, as the assembled TCR complex may associate with endogenous delta, gamma, epsilon and/or zeta chains an ordinary skilled artisan will readily understand that reference to a TCR as present in a cell membrane may include reference to the fully or partially assembled TCR complex as appropriate.

Recombinant or engineered individual TCR chains and TCR complexes have been developed. References to the use of a TCR in a therapeutic context may refer to individual recombinant TCR chains. As such, engineered TCRs may include individual modified TCRα or modified TCRβ chains as well as single chain TCRs that include modified and/or unmodified TCRα and TCRβ chains that are joined into a single polypeptide by way of a linking polypeptide.

As used herein, the term “binding-triggered transcriptional switch” or “BTTS” refers to any polypeptide or complex of the same that is capably of transducing a specific binding event on the outside of the cell (e.g. binding of an extracellular domain of the BTTS) to activation of a recombinant promoter within the nucleus of the cell. Many BTTSs work by releasing a transcription factor that activates the promoter. In these embodiments, the BTTS is made up of one or more polypeptides that undergo proteolytic cleavage upon binding to the antigen to release a gene expression regulator that activates the recombinant promoter. For example, a BTTS may comprise: (i) an extracellular domain comprising the antigen-binding region of an antigen-specific antibody, wherein this region engages with an antigen on another cell; (ii) a transmembrane domain; (iii) an intracellular domain comprising a transcriptional activator; and (iv) one or more proteolytic cleavage sites (e.g., a masked recognition site for an ADAM protease that between the antigen-binding region and the transmembrane domain of the protein, and a site in the transmembrane that is recognized by γ-secretase); where binding of the antigen binding region to the antigen on another cell induces cleavage at the one or more proteolytic cleavage sites, thereby releasing the transcriptional activator. The released transcriptional activator, in turn, activates expression of a downstream protein. A BTTS can be based on synNotch, A2, MESA, or force receptor, for example, although others are known or could be constructed. As such, a BTTS may comprise one or more protease cleavage sites and an intracellular domain comprising a transcriptional activator, wherein binding of the BTTS to the tissue- or a cancer-specific antigen on another cell causes the BTTS to be cleaved at the protease cleavage site, thereby releasing the transcriptional activator, and wherein the released transcriptional activator induces expression of another protein, e.g. an immune receptor or pro-inflammatory protein. In some embodiments, a SNIPR (Zhu et al 2021 bioRxiv) may be used.

The terms “anti-stromal marker binding-triggered transcriptional switch” and “anti-stromal marker BTTS” refer to a binding-triggered transcriptional switch that is activated (i.e., releases the transcription factor) by binding to a stromal marker on another cell, e.g., (i) FAP (Fibroblast Activation Protein Alpha), (ii) PDPN (Podoplanin), (iii) CDH11 (Cadherin 11), (iv) PDGFR (Platelet-derived growth factor) or (v) LRRC15 (Leucine Rich Repeat Containing protein 15). As would be apparent, these BTTSs have a extracellular binding domain, e.g., a scFv or nanobody, that specifically binds to the stromal marker.

A “biological sample” encompasses a variety of sample types obtained from an individual or a population of individuals and can be used in various ways, including e.g., the isolation of cells or biological molecules, diagnostic assays, etc. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by mixing or pooling of individual samples, treatment with reagents, solubilization, or enrichment for certain components, such as cells, polynucleotides, polypeptides, etc. The term “biological sample” encompasses a clinical sample, and also includes cells in culture, cell supernatants, cell lysates, serum, plasma, biological fluid, and tissue samples. The term “biological sample” includes urine, saliva, cerebrospinal fluid, interstitial fluid, ocular fluid, synovial fluid, blood fractions such as plasma and serum, and the like. The term “biological sample” also includes solid tissue samples, tissue culture samples (e.g., biopsy samples), and cellular samples. Accordingly, biological samples may be cellular samples or acellular samples.

The terms “antibodies” and “immunoglobulin” include antibodies or immunoglobulins of any isotype, fragments of antibodies which retain specific binding to antigen, including, but not limited to, Fab, Fv, scFv, and Fd fragments, chimeric antibodies, humanized antibodies, single-chain antibodies, nanobodies, single-domain antibodies, and fusion proteins comprising an antigen-binding portion of an antibody and a non-antibody protein.

“Antibody fragments” comprise a portion of an intact antibody, for example, the antigen binding or variable region of the intact antibody. Examples of antibody fragments include Fab, Fab′, F(ab′)2, and Fv fragments; diabodies; linear antibodies (Zapata et al., Protein Eng. 8(10): 1057-1062 (1995)); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)2 fragment that has two antigen combining sites and is still capable of cross-linking antigen.

“Single-chain Fv” or “sFv” antibody fragments comprise the VH and VL domains of antibody, wherein these domains are present in a single polypeptide chain. In some embodiments, the Fv polypeptide further comprises a polypeptide linker between the VH and VL domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “nanobody” (Nb), as used herein, refers to the smallest antigen binding fragment or single variable domain (V_{HH}) derived from naturally occurring heavy chain antibody and is known to the person skilled in the art. They are derived from heavy chain only antibodies, seen in camelids (Hamers-Casterman et al. (1993) Nature 363:446; Desmyter et al. (2015) Curr. Opin. Struct. Biol. 32:1). In the family of “camelids” immunoglobulins devoid of light polypeptide chains are found. “Camelids” comprise old world camelids (Camelus bactrianus and Camelus dromedarius) and new world camelids (for example, Llama paccos, Llama glama, Llama guanicoe and Llama vicugna). A single variable domain heavy chain antibody is referred to herein as a nanobody or a V_{HH} antibody.

As used herein, the term “affinity” refers to the equilibrium constant for the reversible binding of two agents and is expressed as a dissociation constant (Kd). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding” refers to a direct association between two molecules, due to, for example, covalent, electrostatic, hydrophobic, and ionic and/or hydrogen-bond interactions, including interactions such as salt bridges and water bridges. Non-specific binding would refer to binding with an affinity of less than about 10⁻⁷ M, e.g., binding with an affinity of 10⁻⁶ M, 10⁻⁵ M, 10⁻⁴ M, etc.

A “orthogonal” or “orthogonalized” member or members of a binding pair are modified from their original or wild-type forms such that the orthogonal pair specifically bind one another but do not specifically or substantially bind the non-modified or wild-type components of the pair. Any binding partner/specific binding pair may be orthogonalized, including but not limited to e.g., those binding partner/specific binding pairs described herein.

The terms “domain” and “motif”, used interchangeably herein, refer to both structured domains having one or more particular functions and unstructured segments of a polypeptide that, although unstructured, retain one or more particular functions. For example, a structured domain may encompass but is not limited to a continuous or discontinuous plurality of amino acids, or portions thereof, in a folded polypeptide that comprise a three-dimensional structure which contributes to a particular function of the polypeptide. In other instances, a domain may include an unstructured segment of a polypeptide comprising a plurality of two or more amino acids, or portions thereof, that maintains a particular function of the polypeptide unfolded or disordered. Also encompassed within this definition are domains that may be disordered or unstructured but become structured or ordered upon association with a target or binding partner. Non-limiting examples of intrinsically unstructured domains and domains of intrinsically unstructured proteins are described, e.g., in Dyson & Wright. Nature Reviews Molecular Cell Biology 6:197-208.

The terms “synthetic”, “chimeric” and “engineered” as used herein generally refer to artificially derived polypeptides or polypeptide encoding nucleic acids that are not naturally occurring. Synthetic polypeptides and/or nucleic acids may be assembled de novo from basic subunits including, e.g., single amino acids, single nucleotides, etc., or may be derived from pre-existing polypeptides or polynucleotides, whether naturally or artificially derived, e.g., as through recombinant methods. Chimeric and engineered polypeptides or polypeptide encoding nucleic acids will generally be constructed by the combination, joining or fusing of two or more different polypeptides or polypeptide encoding nucleic acids or polypeptide domains or polypeptide domain encoding nucleic acids. Chimeric and engineered polypeptides or polypeptide encoding nucleic acids include where two or more polypeptide or nucleic acid “parts” that are joined are derived from different proteins (or nucleic acids that encode different proteins) as well as where the joined parts include different regions of the same protein (or nucleic acid encoding a protein) but the parts are joined in a way that does not occur naturally.

The term “recombinant”, as used herein describes a nucleic acid molecule, e.g., a polynucleotide of genomic, cDNA, viral, semisynthetic, and/or synthetic origin, which, by virtue of its origin or manipulation, is not associated with all or a portion of the polynucleotide sequences with which it is associated in nature. The term recombinant as used with respect to a protein or polypeptide means a polypeptide produced by expression from a recombinant polynucleotide. The term recombinant as used with respect to a host cell or a virus means a host cell or virus into which a recombinant polynucleotide has been introduced. Recombinant is also used herein to refer to, with reference to material (e.g., a cell, a nucleic acid, a protein, or a vector) that the material has been modified by the introduction of a heterologous material (e.g., a cell, a nucleic acid, a protein, or a vector).

The term “operably linked” refers to a juxtaposition wherein the components so described are in a relationship permitting them to function in their intended manner. For instance, a promoter is operably linked to a coding sequence if the promoter affects its transcription or expression. Operably linked nucleic acid sequences may but need not necessarily be adjacent. For example, in some instances a coding sequence operably linked to a promoter may be adjacent to the promoter. In some instances, a coding sequence operably linked to a promoter may be separated by one or more intervening sequences, including coding and non-coding sequences. Also, in some instances, more than two sequences may be operably linked including but not limited to e.g., where two or more coding sequences are operably linked to a single promoter.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

A “vector” or “expression vector” is a replicon, such as plasmid, phage, virus, or cosmid, to which another DNA segment, i.e. an “insert”, may be attached so as to bring about the replication of the attached segment in a cell.

The term “heterologous”, as used herein, means a nucleotide or polypeptide sequence that is not found in the native (e.g., naturally-occurring) nucleic acid or protein, respectively. Heterologous nucleic acids or polypeptide may be derived from a different species as the organism or cell within which the nucleic acid or polypeptide is present or is expressed. Accordingly, a heterologous nucleic acids or polypeptide is generally of unlike evolutionary origin as compared to the cell or organism in which it resides.

The term “activates” in the context of activating expression of the pro-inflammatory protein, means inducing the transcription, translation and secretion of the pro-inflammatory cytokine.

The term “cancer-associated” refers to an antigen that is expressed in cancerous cells but not significantly non-cancerous cells of the same type. Some cancer-associated antigens are expressed on cancer cells and in normal tissues. For example, MSLN is considered a cancer-associated antigen since it is aberrantly expressed various cancer cells (e.g., lung cancers (adenocarcinoma and squamous carcinoma), ovary, peritoneum, endometrium, pancreas, stomach and colon, etc.) but it is also expressed on normal mesothelial cells in the pleura, pericardium, and peritoneum and in epithelial cells on the surface of the ovary, tunica vaginalis, rete testis, and fallopian tubes in trace amounts.

The term “activates” or “activated by” in the context of a CAR or BTTS, means that the CAR or BBTS is activated by binding to one or more antigens on another cell or to multiple different antigens on different cells, where the antigens may be selected from Table 1.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a cell” includes a plurality of such cells and reference to “the cell” includes reference to one or more cells and equivalents thereof known to those skilled in the art, and so forth. For instance, an immune receptor that is activated by binding to “a” cancer-associated antigen in a solid tumor could be activated by binding to a single target antigen or to two target antigens (in the case of a tandem CAR and the like). It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

As summarized above, the present disclosure provides a cytotoxic immune cell that is primed by and/or whose cytotoxicity within the tumor microenvironment is enhanced by binding to a stromal marker.

In some embodiments, the cell comprises a molecular circuit comprising the following components: (a) an anti-stromal marker binding-triggered transcriptional switch (BTTS) and one or both of: (b) a nucleic acid encoding a pro-inflammatory protein, and (c) a nucleic acid encoding a immune receptor (e.g., a CAR or engineered TCR) that is activated by binding to an antigen listed in Table 1 on a cancer cell, wherein binding of the BTTS to the stromal marker on the surface of a stromal cell activates expression of the pro-inflammatory protein and/or the immune receptor. The cell may comprise components (a), (b) and (c), components (a) and (b), or components (a) and (c).

In other embodiments, the cell may comprise the following components: (a) an anti-stromal marker BTTS, (b) a nucleic acid encoding a pro-inflammatory protein, wherein binding of the BTTS to FAP on the surface of a stromal cell activates expression of the pro-inflammatory protein of (b); and (c) the immune receptor that is activated by binding to an antigen listed in Table 1 on a cancer cell. In these embodiments, expression of the immune receptor may be constitutive in the cell.

In any embodiment, the stromal marker may be: (i) FAP (Fibroblast Activation Protein Alpha), (ii) PDPN (Podoplanin), (iii) CDH11 (Cadherin 11), (iv) PDGFR (Platelet-derived growth factor) or (v) LRRC 15 (Leucine Rich Repeat Containing protein 15) Cancer-associated antigens in solid tumors to which the BTTS may bind are listed in Table 1 below.

TABLE 1

| | | |
|---|---|---|
| Mesothelin | Trop2 (TACSTD2) | KREMEN2 |
| EGFRvIII | MUC1 | GRIN2D |
| IL13RA2 | ROR1 | CELSR3 |
| EPHA2 | Claudin18.2 | KISS1R |
| PSMA (FOLH1) | GPC3 | CDH6 |
| HER2 | CA9 | TREM1 |
| EGFR | EPCAM | MC1R |
| PSCA | FOLR1 | LIFR |
| ALPPL2 | L1CAM | PRR7 |
| GD2 (B4GALNT1) | CD133 (PROM1) | PTPRN |
| BCAN | CEA (CEACAM5) | CACNG7 |
| MOG | MUC16 | TRPM1 |
| CSPG5 | PDL1 (CD274) | |
| CD70 | ROR2 | |
| MET | VEGFR2 (KDR) | |
| AXL | CD44v6 | |
| MCAM | NKG2D (KLRK1) | |
| DLL3, DLL4 | MLANA (MART1) | |
| nectin4 | NY-ESO | |
| nectin2 | TYRP-1 | |
| nectin3 | AFP | |
| nectin1 | MAGE family* | |
| ALK | WT-1 | |

*Reference to “MAGE family” includes any of the MAGE family members listed in Table 2 of Weon et al (Curr Opin Cell Biol. 2015 37: 1-8), particularly MAGE A1, MAGE A2, MAGE A3, MAGE A4, which are each associated with various solid tumors, e.g., NSCLC, melanoma, breast, ovarian and colon.

In some embodiments, an antigen may be selected from the following list: mesothelin, FAP, EGFRvIII, IL13RA2, EPHA2, PSMA (FOLH1), HER2, EGFR, PSCA, ALPPL2, GD2 (B4GALNT1), BCAN, MOG, CSPG5, CD70, MET, AXL, MCAM, DLL3, DLL4, nectin4, nectin2, nectin3, nectin1, and ALK.

### Cells

The cells employed herein are immune cells that contain one or more of the described nucleic acids, expression vectors, etc., encoding the desired components. Immune cells of the present disclosure include mammalian immune cells including, e.g., those that are genetically modified to produce the components of a circuit of the present disclosure or to which a nucleic acid, as described above, has been otherwise introduced. In some instances, the subject immune cells have been transduced with one or more nucleic acids and/or expression vectors to express one or more components of a circuit of the present disclosure.

Suitable mammalian immune cells include primary cells and immortalized cell lines. Suitable mammalian cell lines include human cell lines, non-human primate cell lines, rodent (e.g., mouse, rat) cell lines, and the like. In some instances, the cell is not an immortalized cell line, but is instead a cell (e.g., a primary cell) obtained from an individual. For example, in some cases, the cell is an immune cell, immune cell progenitor or immune stem cell obtained from an individual. As an example, the cell is a lymphoid cell, e.g., a lymphocyte, or progenitor thereof, obtained from an individual. As another example, the cell is a cytotoxic cell, or progenitor thereof, obtained from an individual.

Such cells include, e.g., lymphoid cells, i.e., lymphocytes (T cells, B cells, natural killer (NK) cells), and myeloid-derived cells (neutrophil, eosinophil, basophil, monocyte, macrophage, dendritic cells). “T cell” includes all types of immune cells expressing CD3 including T-helper cells (CD4+ cells) and cytotoxic T-cells (CD8+ cells). A “cytotoxic cell” includes CD8+ T cells, natural-killer (NK) cells, and neutrophils, which cells are capable of mediating cytotoxicity responses.

Immune cells encoding a circuit of the present disclosure may be generated by any convenient method. Nucleic acids encoding one or more components of a subject circuit may be stably or transiently introduced into the subject immune cell, including where the subject nucleic acids are present only temporarily, maintained extrachromosomally, or integrated into the host genome. Introduction of the subject nucleic acids and/or genetic modification of the subject immune cell can be carried out in vivo, in vitro, or ex vivo.

In some cases, the introduction of the subject nucleic acids and/or genetic modification is carried out ex vivo. For example, a primary T lymphocyte, a stem cell, or an NK cell is obtained from an individual; and the cell obtained from the individual is modified to express components of a circuit of the present disclosure.

### Anti-Stromal Marker Binding-Triggered Transcriptional Switches

The anti-stromal marker BTTS is a cleavable fusion protein contains: (a) an extracellular binding domain comprising a protein binding domain (e.g., scFv or nanobody) that binds to the stromal maker, (b) a force sensing region, (c) a transmembrane domain, (d) one or more force-dependent cleavage sites that are cleaved when the force sensing region is activated, and (e) an intracellular domain comprising a transcriptional activator, where binding of the binding domain to the stromal markr on the surface of a cell induces proteolytic cleavage of the one or more force-dependent cleavage sites to release the transcriptional activator. The stromal marker may be (i) FAP (Fibroblast Activation Protein Alpha), (ii) PDPN (Podoplanin), (iii) CDH11 (Cadherin 11), (iv) PDGFR (Platelet-derived growth factor) or (v) LRRC15 (Leucine Rich Repeat Containing protein 15).

In this switch, the fusion protein is cleaved to release the intracellular domain when the extracellular domain of the fusion protein engages with a stromal marker on another cell. As such, in many cases, the fusion protein will contain a force sensing region (which is typically in the extracellular domain) and one or more force-dependent cleavage sites that are cleaved when the force sensing region is activated. The position of the force-dependent cleavage sites may vary and, in some embodiments the fusion protein may contain at least two cleavage sites. In some cases, one of the cleavage sites may be extracellular and the other may be in the transmembrane domain or within 10 amino acids of the transmembrane domain in the intracellular domain. In any embodiment, the force sensing region and/or the one or more force-dependent cleavage sites may be from a Delta/Serrate/Lag2 (DSL) superfamily protein, as reviewed by Pintar et al (Biology Direct 2007 2: 1-13). For example, the force sensing region and/or the one or more force-dependent cleavage sites may be from Notch (see Morsut Cell. 2016 164: 780-91), von Willebrand Factor (vWF), amyloid-beta, CD16, CD44, Delta, a cadherin, an ephrin-type receptor or ephrin ligand, a protocadherin, a filamin, a synthetic E cadherin, interleukin-1 receptor type 2 (IL1R2), major prion protein (PrP), a neuregulin or an adhesion-GPCR. Several other examples of this type of protein are known and listed in Pintar, supra. Many members of this family appear to share a similar architecture a region that unfolds and opens up a protease cleavage site (e.g., EGF-like repeats; see Cordle et al Nat. Struct. Mol. Biol. 2008 15: 849-857), a trans-membrane segment, and a relatively short (˜100-150 amino acids) intracellular domain. These sequences permit the binding-triggered release of a transcriptional activator from the membrane in their natural environment and can be readily adapted herein.

In some cases, the one or more ligand-inducible proteolytic cleavage sites are selected from S1, S2, and S3 proteolytic cleavage sites. In some cases, the S1 proteolytic cleavage site is a furin-like protease cleavage site comprising the amino acid sequence Arg-X-(Arg/Lys)-Arg, where X is any amino acid. In some cases, the S2 proteolytic cleavage site ADAM-17-type protease cleavage site comprising an Ala-Val dipeptide sequence. In some cases, the S3 proteolytic cleavage site is a γ-secretase cleavage site comprising a Gly-Val dipeptide sequence. The S3 proteolytic cleavage site is in the transmembrane domain. In many cases, the shear force generated by binding of the extracellular domain of this fusion protein to another cells unfolds the force sensing region (which, in the case of Notch contains EGF-like repeats whereas in other protein is made up of other sequences such as the A2 domain in vWF (see, e.g., J Thromb Haemost. 2009 7:2096-105, Lippok Biophys J. 2016 110: 545-54, Lynch Blood. 2014 123: 2585-92, Crawley, Blood. 2011 118:3212-21 and Xy J Biol Chem. 2013 288:6317-24) or modified A2 domain that has, e.g., the R1597W, E1638K and I1628T substitutions. The architecture of such proteins is described in, e.g., Morsut et al, Cell. 2016 164: 780-91, WO2016138034 and WO2019099689, among other places).

In some cases, the fusion protein includes an S1 ligand-inducible proteolytic cleavage site. An S1 ligand-inducible proteolytic cleavage site can be located between the HD-N segment and the HD-C segment. In some cases, the S1 ligand-inducible proteolytic cleavage site is a furin-like protease cleavage site. A furin-like protease cleavage site can have the canonical sequence Arg-X-(Arg/Lys)-Arg, where X is any amino acid; the protease cleaves immediately C-terminal to the canonical sequence. For example, in some cases, an amino acid sequence comprising an S1 ligand-inducible proteolytic cleavage site can have the amino acid sequence GRRRRELDPM (SEQ ID NO: 31), where cleavage occurs between the “RE” sequence. As another example, an amino acid sequence comprising an S1 ligand-inducible proteolytic cleavage site can have the amino acid sequence RQRRELDPM (SEQ ID NO: 32), where cleavage occurs between the “RE” sequence.

In some cases, the fusion protein polypeptide includes an S2 ligand-inducible proteolytic cleavage site. An S2 ligand-inducible proteolytic cleavage site can be located within the HD-C segment. In some cases, the S2 ligand-inducible proteolytic cleavage site is an ADAM-17-type protease cleavage site. An ADAM-17-type protease cleavage site can comprise an Ala-Val dipeptide sequence, where the enzyme cleaves between the Ala and the Val. For example, in some cases, amino acid sequence comprising an S2 ligand-inducible proteolytic cleavage site can have the amino acid sequence KIEAVKSE (SEQ ID NO: 33), where cleavage occurs between the “AV” sequence. As another example, an amino acid sequence comprising an S2 ligand-inducible proteolytic cleavage site can have the amino acid sequence KIEAVQSE (SEQ ID NO: 34), where cleavage occurs between the “AV” sequence.

In some cases, the fusion protein includes an S3 ligand-inducible proteolytic cleavage site. An S3 ligand-inducible proteolytic cleavage site can be located within the TM domain. In some cases, the S3 ligand-inducible proteolytic cleavage site is a gamma-secretase (γ-secretase) cleavage site. A γ-secretase cleavage site can comprise a Gly-Val dipeptide sequence, where the enzyme cleaves between the Gly and the Val. For example, in some cases, an S3 ligand-inducible proteolytic cleavage site has the amino acid sequence VGCGVLLS (SEQ ID NO: 35), where cleavage occurs between the “GV” sequence. In some cases, an S3 ligand-inducible proteolytic cleavage site comprises the amino acid sequence GCGVLLS (SEQ ID NO: 36).

In some cases, the fusion protein polypeptide lacks an S1 ligand-inducible proteolytic cleavage site. In some cases, the Notch receptor polypeptide lacks an S2 ligand-inducible proteolytic cleavage site. In some cases, the Notch receptor polypeptide lacks an S3 ligand-inducible proteolytic cleavage site. In some cases, the Notch receptor polypeptide lacks both an S1 ligand-inducible proteolytic cleavage site and an S2 ligand-inducible proteolytic cleavage site. In some cases, the Notch receptor polypeptide includes an S3 ligand-inducible proteolytic cleavage site; and lacks both an S1 ligand-inducible proteolytic cleavage site and an S2 ligand-inducible proteolytic cleavage site.

In other embodiments, the fusion protein may have an vWF A2 sequence or a variation thereof, an ADAMTS13 cleavage site (which may be described by the consensus sequence HEXXHXXGXXHD; Crawley, Blood. 2011 118:3212-21), and an S3 or γ-secretase cleavage site, although many other arrangements exist. In some embodiments, the switch may contain components that are borrowed from Notch. In other embodiments, the switch may not contain components that are from Notch.

In some embodiments, the transmembrane domain of the fusion protein may contain a γ-secretase cleavage site comprising a Gly-Val dipeptide sequence, since Zhu et al (2021 bioRxiv) has shown that the SNIPRs (which are a type of BTTS) that have a transmembrane domain that contains a γ-secretase cleavage site do not require an ADAM cleavage site.

For simplicity, BTTSs, including but not limited to chimeric notch receptor polypeptides, are described primarily as single polypeptide chains. However, BTTSs, including chimeric notch receptor polypeptides, may be divided or split across two or more separate polypeptide chains where the joining of the two or more polypeptide chains to form a functional BTTS, e.g., a chimeric notch receptor polypeptide, may be constitutive or conditionally controlled. For example, constitutive joining of two portions of a split BTTS may be achieved by inserting a constitutive heterodimerization domain between the first and second portions of the split polypeptide such that upon heterodimerization the split portions are functionally joined.

Useful BTTSs that may be employed in the subject methods include, but are not limited to modular extracellular sensor architecture (MESA) polypeptides. A MESA polypeptide comprises: a) a ligand binding domain; b) a transmembrane domain; c) a protease cleavage site; and d) a functional domain. The functional domain can be a transcription regulator (e.g., a transcription activator, a transcription repressor). In some cases, a MESA receptor comprises two polypeptide chains. In some cases, a MESA receptor comprises a single polypeptide chain. Non-limiting examples of MESA polypeptides are described in, e.g., U.S. Patent Publication No. 2014/0234851; the disclosure of which is incorporated herein by reference in its entirety. In some embodiments, a SNIPR (Zhu et al 2021 bioRxiv) may be used.

Useful BTTSs that may be employed in the subject methods include, but are not limited to polypeptides employed in the TANGO assay. The subject TANGO assay employs a TANGO polypeptide that is a heterodimer in which a first polypeptide comprises a tobacco etch virus (Tev) protease and a second polypeptide comprises a Tev proteolytic cleavage site (PCS) fused to a transcription factor. When the two polypeptides are in proximity to one another, which proximity is mediated by a native protein-protein interaction, Tev cleaves the PCS to release the transcription factor. Non-limiting examples of TANGO polypeptides are described in, e.g., Barnea et al. (Proc Natl Acad Sci USA. 2008 Jan. 8; 105(1):64-9); the disclosure of which is incorporated herein by reference in its entirety.

Useful BTTSs that may be employed in the subject methods include, but are not limited to von Willebrand Factor (vWF) cleavage domain-based BTTSs, such as but not limited to e.g., those containing a unmodified or modified vWF A2 domain. A subject vWF cleavage domain-based BTTS will generally include: an extracellular domain comprising a first member of a binding pair; a von Willebrand Factor (vWF) cleavage domain comprising a proteolytic cleavage site; a cleavable transmembrane domain and an intracellular domain. Non-limiting examples of vWF cleavage domains and vWF cleavage domain-based BTTSs are described in Langridge & Struhl (Cell (2017) 171(6):1383-1396); the disclosure of which is incorporated herein by reference in its entirety.

Useful BTTSs that may be employed in the subject methods include, but are not limited to chimeric Notch receptor polypeptides, such as but not limited to e.g., synNotch polypeptides, non-limiting examples of which are described in PCT Pub. No. WO 2016/138034, U.S. Pat. Nos. 9,670,281, 9,834,608, Roybal et al. Cell (2016) 167(2):419-432, Roybal et al. Cell (2016) 164(4):770-9, and Morsut et al. Cell (2016) 164(4):780-91; the disclosures of which are incorporated herein by reference in their entirety.

Anti-FAP antibodies that could be employed in the present fusion protein are numerous and include those described by Mersmann et al (Int J Cancer 2001 92: 240-8), Zhang et al (FASEB J. 2013 27: 581-589), Brocks et al (Molecular Medicine 2001 7: 461-469), Schmidt et al (European Journal of Biochemistry 2001 268:1730-8) WO2016110598, WO2016116399, WO2014055442, US20090304718 and U.S. Pat. No. 10,253,110, which are incorporated by reference for a description of at least the CDRs of those antibodies.

In some embodiments, an anti-FAP antibody may have HC and LC CDR1, 2 and 3 sequences that are identical to or similar (i.e., may contain up to 5 amino acid substitutions, e.g., up to 1, up to 2, up to 3, up to 4 or up to 5 amino acid substitutions, collectively) to the CDRs of any of SEQ ID NOS 1-8, shown below, which CDRs are indicated. The framework sequence could be humanized, for example. In some embodiments, the anti-FAP antibody may have HC and LC variable regions that are at least 90%, at least 95%, at least 98% or at least 99% identical to a pair of HC and LC sequences (SEQ ID NOS 1-8)shown below.

Expression of the anti-stromal marker BTTS in the cell may be constitutive or inducible, e.g., by binding of another BTTS to an antigen on another cell in the tumor.

Examples of transcriptional activators that can be part of the fusion protein are numerous and include artificial transcription factors (ATFs) such as, e.g., Zinc-finger-based artificial transcription factors (including e.g., those described in Sera T. Adv Drug Deliv Rev. 2009 61(7-8):513-26; Collins et al. Curr Opin Biotechnol. 2003 14(4):371-8; Onori et al. BMC Mol Biol. 2013 14:3. In some cases, the transcriptional activator may contain a GAL4 DNA binding domain, which binds to the Gal4 responsive UAS, which has been well characterized in the art. Examples of suitable transcriptional activators include GAL4-VP16 and GAL4-VP64, although many others could be used. As would be appreciated, the identity of the transcription activators may vary. In some embodiments, the transcription factor may have a DNA binding domain that binds to a corresponding promoter sequence and an activation domain. In many embodiments, the DNA binding domain of the first and second transcription factors may be independently selected from Gal4-, LexA-, Tet-, Lac-, dCas9-, zinc-finger- and TALE-based transcription factors. TALE- and CRISPR/dCas9-based transcription factors are described in Lebar (Methods Mol Biol. 2018 1772: 191-203), among others. The binding sites for such domains are well known or can be designed at will. The first and second transcription factors can have any suitable activation domain, e.g., VP16, VP64, Ela, Spl, VP16, CTF, GAL4 among many others.

### Circuits

As noted above, binding of BTTS the stromal marker on the surface of a stromal cell activates expression of another protein, e.g., a CAR and/or pro-inflammatory protein. In these embodiments, binding of the binding domain of the BTTS to the stromal marker on the surface of a stromal cell induces proteolytic cleavage of the one or more force-dependent cleavage sites to release the transcriptional activator. The released transcriptional activator then binds to a promoter that drives the expression of the immune receptor and/or pro-inflammatory protein, thereby inducing expression of the immune receptor and/or pro-inflammatory protein. The general principles of a circuit are described in WO 2016/138034, U.S. Pat. Nos. 9,670,281, 9,834,608, Roybal et al. Cell (2016) 167(2):419-432, Roybal et al. Cell (2016) 164(4):770-9, and Morsut et al. Cell (2016) 164(4):780-91, among others.

### Cytotoxic Immune Receptors

As noted above, in any embodiment the immune receptor may be a chimeric antigen receptor (CAR) or an engineered T cell receptor (TCR). In any embodiment, the immune receptor may be constitutively expressed. In other embodiments, expression of the immune receptor may be under the control of another BTTS. In either event, the immune receptor will be expressed on the surface of the cell and will be activated by binding to an antigen that is expressed by the cancerous cells, e.g., by the malignant cells, e.g., an antigen listed in Table 1, for example. Binding domains for many of these antigens are described above.

As noted above, in some embodiments the BTTS may induce expression of an immune receptor. In these embodiments, the immune receptor may be induced by the BTTS binding to the stromal marker. In other embodiments, the immune receptor may be constitutively expressed. In either event, the immune receptor will be expressed on the surface of the cell and will be activated by binding to an antigen that is expressed by the malignant cells, e.g., an antigen listed in Table 1.

Binding of the immune receptor its cognate antigen activates the immune cell. In these embodiments, the circuit may comprise a nucleic acid containing a promoter that is activated by the released transcriptional activator, and a coding sequence encoding a pro-inflammatory protein. Immune receptors can be designed in several ways (see, generally, e.g., Guedan et al, Methods and Clinical Development 2019 12: 145-156) and in soe embodiments may include an extracellular domain that contains an antigen binding domain such as a scFv or nanobody, a hinge, a transmembrane region (which may be derived from CD4, CD8α, or CD28), a costimulatory signaling domains (which may be derived from the intracellular domains of the CD28 family (e.g., CD28 and ICOS) or the tumor necrosis factor receptor (TNFR) family of genes (e.g., 4-1BB, OX40, or CD27), and an ITAM domain, e.g., the signaling domain from the zeta chain of the human CD3 complex (CD3zeta). In practice, any of these domains may be a variation of a wild type sequence. In practice, any of these sequences may be a variant of a wild type sequence, e.g., a sequence that is at least 90%, 95, or 98% identical a sequence described in WO2014127261, for example.

The binding domain of the immune receptor may be specific for an antigen listed in Table 1. In some embodiments, a binding domain of the immune receptor may have HC and LC CDR1, 2 and 3 sequences that are identical to or similar (i.e., may contain up to 5 amino acid substitutions, e.g., up to 1, up to 2, up to 3, up to 4 or up to 5 amino acid substitutions, collectively) to the CDRs of any of the antibodies listed in the publication cited in the table below, which publications are incorporated by reference for those sequences. The framework sequence could be humanized, for example. In some embodiments, the binding domain of the immune receptor may have HC and LC variable regions that are at least 90%, at least 95%, at least 98% or at least 99% identical to a pair of HC and LC sequences listed in the publication cited in the table below, which publications are incorporated by reference for those sequences.

| Antigen | |
|---|---|
| binding | |
| domain | Exemplary sources of antigen binding sequences |
| Mesothelin | US 2021/0290676, US 2021/0284728 A1, US 2021/ |
| (MSLN) | 0275584 A1, Feng et al., Mol. Cancer Ther. 8(5): |
| | 1113-1118 (2009), US 2021/0269537 A1, US 2021/ |
| | 0252122 A1, US 2021/0230242 A1, US 2021/0155702 A1, |
| | US 2021/0137977 A1, US 2021/01016620 A1 |
| FAP | US 2021/0252122 A1, Kakarla et al. Mol Ther. 2013 |
| | August; 21(8): 1611-20, Wang et al. Cancer Immunol Res. |
| | 2015 July; 3(7): 815-826, Petrausch et al. BMC Cancer. |
| | 2012; 12: 615, Tran et al. J Exp Med. 2013 Jun. 3; |
| | 210(6): 1125-35. |
| Her2 | US 2021/0299269, US 2021/0290676, US 2021/0137977 |
| | A1, US 2021/01016620 A1, US 2021/0299172 A1 |
| Trop2 | US 2021/0290676, Zhao et al. Am J Cancer Res. 2019; |
| | 9(8): 1846-1856., Bedoya et al. Cytotherapy 2019 May; |
| | 21(5): S11-12., Sayama et al. Mol Med Rep. 2021 |
| | February; 23(2): 92. |
| GPC3 | US 2021/0261646 A1, US 2021/0137977 A1, US 2021/ |
| | 01016620 A1, Li et al. Am J Transl Res. 2021 |
| | Jan. 15; 13(1): 156-167., Batra et al. Cancer |
| | Immunol Res. 2020 March; 8(3): 309-320. |
| MUC1 | US 2021/0269547 A1, US 2021/0155702 A1, Supimon |
| | et al. Sci Rep. 2021 Mar. 18; 11(1): 6276., Zhou |
| | et al. Front Immunol. 2019 May 24; 10: 1149., |
| | Mei et al. Cancer Med. 2020 January; 9(2): 640-652. |
| ROR1 | US 2021/0290676, Wallstabe et al JCI Insight. 2019 |
| | Sep. 19; 4(18): e126345, US 2021/0137977 A1, Prussak |
| | et al. J. Clin. Oncol. 2020; 38, no. 6_suppl, Srivastava |
| | et al. Cancer Cell. 2021 Feb. 8; 39(2): 193-208.e10. |
| EPCAM | US 2021/0290676, US 2021/0284728 A1, US 2021/ |
| | 0269547 A1, Qin et al. Oncoimmunology. 2020 Aug. |
| | 15; 9(1): 1806009., Deng et al. BMC Immunol. 2015 |
| | Jan. 31; 16(1): 1. |
| ALPPL2 | Su et al Cancer Res. 2020 Oct. 15; 80(20): 4552-4564., |
| | Hyrenius-Wittsten et al. Sci Transl Med. 2021 Apr. 28; |
| | 13(591): eabd8836., WO2017095823A1 |
| PSMA | US 2021/0290676, US 2021/0284728 A1, US 2021/ |
| | 0269547 A1, US 2021/0252122 A1, US 2021/0137977 A1, |
| | US 2021/0113615 A1 |
| PSCA | US 2021/0290676, US 2021/0269547 A1, Wu et al. |
| | Biomark Res. 2020 Jan. 28; 8: 3., Dorff et al. J. Clin. |
| | Oncol. 2020; 38, no. 6_suppl, US 2020/0308300 |
| EGFRviii | US 2021/0290676, US 2021/0252122 A1, US 2021/ |
| | 0137977 A1, O'Rourke et al. Sci Transl Med. (2017) |
| | 9: eaaa0984, Abbott et al. Clin Transl Immunology. |
| | 2021 May 9; 10(5): e1283. |
| EGFR | US 2021/0290676, US 2021/0269547 A1, US 2021/ |
| | 0155702 A1, Xia et al. Clin Transl Immunology. |
| | 2020 May 3; 9(5): e01135., Li et al. Cell Death Dis. |
| | 2018 February; 9(2): 177., Liu et al. Clinical Trial |
| | Cytotherapy. 2020 October; 22(10): 573-580. |
| Claudin 18.2 | US 2021/0252122 A1, Jiang et al. J Natl Cancer Inst. |
| | 2019 Apr. 1; 111(4): 409-418., Chin et al. J Cancer |
| | Res. 2020 April; 32(2): 263-270., Zhan et al. J. Clin. |
| | Oncol. 2019, 37, 2509., Singh et al. J Hematol Oncol. |
| | 2017; 10: 105. |
| GD2 | US 2021/0290676, Seitz et al. Oncoimmunology. 2020; |
| | 9(1): 1683345., Chulanetra et al. Am J Cancer Res. |
| | 2020; 10(2): 674-687., Sujjitjoon et al. Transl Oncol. |
| | 2021 February; 14(2): 100971, Andersch et al. BMC |
| | Cancer. 2019 Sep. 9; 19(1): 895. |

However, sequences that bind to other antigens are known and/or can be readily made.

New antigen binding domains may also be generated in the form of immunoglobulin single variable (ISV) domains. The ISV domains may be generated using any suitable method. Suitable methods for the generation and screening of ISVs include without limitation, immunization of dromedaries, immunization of camels, immunization of alpacas, immunization of sharks, yeast surface display, etc. Yeast surface display has been successfully used to generate specific ISVs as shown in McMahon et al. (2018) Nature Structural Molecular Biology 25(3): 289-296 which is specifically incorporated herein by reference.

Immunoglobulin sequences, such as antibodies and antigen binding fragments derived there from (e.g., immunoglobulin single variable domains or ISVs) are used to specifically target the respective antigens disclosed herein. The generation of immunoglobulin single variable domains such as e.g., VHHs or ISV may involve selection from phage display or yeast display, for example ISV can be selected by utilizing surface display platforms where the cell or phage surface display a synthetic library of ISV, in the presence of tagged antigen. A fluorescent secondary antibody directed to the tagged antigen is added to the solution thereby labeling cells bound to antigen. Cells are then sorted using any cell sorting platform of interest e.g., magnetic-activated cell sorting (MACS) or fluorescence-activated cell sorting (FACS). Sorted clones are amplified, resulting in an enriched library of clones expressing ISV that bind antigen. The enriched library is then re-screened with antigen to further enrich for surface displayed antigen binding ISV. These clones can then be sequenced to identify the sequences of the ISV of interest and further transferred to other heterologous systems for large scale protein production.

In some embodiments, an anti-MSLN antibody may have HC and LC CDR1, 2 and 3 sequences that are identical to or similar (i.e., may contain up to 5 amino acid substitutions, e.g., up to 1, up to 2, up to 3, up to 4 or up to 5 amino acid substitutions, collectively) to the CDRs of any of SEQ ID NOS 9-15, shown below, which CDRs are indicated. The framework sequence could be humanized, for example. In some embodiments, the anti-MSLN antibody may have HC and LC variable regions that are at least 90%, at least 95%, at least 98% or at least 99% identical to a pair of HC and LC sequences (SEQ ID NOS 9-15) shown below.

### Pro-Inflammatory Proteins

As noted above, binding of BTTS to the stromal marker on the surface of another cell activate expression of a pro-inflammatory protein and/or immune receptor. In these embodiments, binding of the binding domain of the BTTS to stromal markerin the tumor induces proteolytic cleavage of the one or more force-dependent cleavage sites to release the transcriptional activator. The released transcriptional activator then binds to a promoter that drives the expression of the pro-inflammatory protein and or/immune receptor. The general principles of a circuit are described in WO 2016/138034, U.S. Pat. Nos. 9,670,281, 9,834,608, Roybal et al. Cell (2016) 167(2):419-432, Roybal et al. Cell (2016) 164(4):770-9, and Morsut et al. Cell (2016) 164(4):780-91, among others.

The pro-inflammatory protein will be secreted from the cell. In these embodiments, the circuit may comprise a nucleic acid containing a promoter that is activated by the released transcriptional activator, and a coding sequence encoding a pro-inflammatory protein. In this disclosure, the term “pro-inflammatory protein” is intended to encompass any cytokine that have a pro-inflammatory activity (e.g., IL-2, CCL-21, IL-12, IL-7, IL-15 and IL-21, etc.), as well as non-natural or “engineered” cytokines that have pro-inflammatory activity such as super IL-2 (see, e.g., Levin et al Nature 2012 484: 529-533), mini-TGF-Beta (which blocks TGF-Beta signaling) and DR-18 (an IL-18 variant), etc.). Engineered cytokines include superkines, which often have up to 10 amino acid substitutes relative to a natural cytokine, as well as natural cytokines that have been truncated, and dominant variants. Cytokines of interest include selected from IL-2, IL-12, IL-15, IL-7, CD40L, or a non-natural variant of IL-2, IL-12, IL-15, IL-7, CD40L that has pro-inflammatory activity. Cytokines include “ortho” cytokines that can be paired with a receptor in the immune cell (see, e.g., Sockolosky et al. 2018). Other pro-inflammatory proteins include immune checkpoint inhibitors, including molecules that block interactions with PD1, CTLA4, BTLA, CD160, KRLG-1, 2B4, Lag-3, Tim-3 and other immune checkpoints. See, e.g., Odorizzi and Wherry (2012) J. Immunol. 188:2957; and Baitsch et al. (2012) PLoSOne 7: e30852. For example, an anti-PD1 antibody, an anti-PDL1 antibody, a decoy resistant IL-18, a dominant negative TGF-β, a dominant negative TGFβ receptor, or a TGFβ inhibitor/agonist could be used. In some embodiments, activation of the circuit may induce the express of a combination of pro-inflammatory proteins.

Sources for exemplary pro-inflammatory proteins are listed below.

| Payload | Exemplary sources of payload sequences |
|---|---|
| IL2 | NCBI Gene ID: 3558 |
| super-2 | Levin et al. Nature. 2012 Apr. 26; 484(7395): |
| | 529-533. |
| ortho2 | Sockolosky et al. Science. 2018 Mar. 2; 359(6379): |
| | 1037-1042. |
| IL15 | NCBI Gene ID: 3600 |
| Anti-PD1 | Pembrolizumab, Nivolumab, Cemiplimab, |
| | Dostarlimab, JTX-4014, Spartalizumab, |
| | Camrelizumab, Sintilimab, Tislelizumab, |
| | Toripalimab, INCMGA00012, AMP-224, AMP-514 |
| Anti-PDL1 | Atezolizumab, Avelumab, Durvalumab, KN035, |
| | CK-301, AUNP12, CA-170, BMS-986189 |
| IL7 | NCBI Gene ID: 3574 |
| CD40L | NCBI Gene ID: 959 |
| Decoy Resistant | Zhou et al. Nature. 2020 July; 583(7817): 609-614. |
| IL-18 (DRIL-18) | |
| Dominant | Kloss et al. Mol Ther. 2018 Jul. 5; 26(7): 1855-1866., |
| negative | Wieser et al. Mol Cell Biol. 1993 December; 13(12): |
| TGFb | 7239-47. |

As would be appreciated, pro-inflammatory proteins are secreted from the cell and their coding sequence will encode a secretion signal.

As noted above, in some embodiments the immune cell may additionally express a recombinant receptor for the pro-inflammatory protein, which further enhances the immune cell's response. For example, if the pro-inflammatory protein is an “ortho2”, then the immune cell may additionally express a receptor for that pro-inflammatory protein.

### Methods of Treatment

A method of treatment for a cancers that are positive for a stromal marker is described below. In general terms, this method may comprise administering a cell described above to the subject. In some embodiments, primary immune cells (e.g., T cells, macrophages or NK cells, etc.) may be purified from an individual, constructs encoding the above proteins may be introduced into the cells ex vivo, and the recombinant cells may be expanded and administered to the subject, e.g., by injection. In other embodiments, allogeneic cells may be used.

The antigen targeted by the immune receptor depends on which cancer is being stromal marker+ cancer is treated. The following table provides a list of stromal cancers and the antigens that are frequently expressed by those tumors. Selection of the binding sequences for the immune receptor may be based on Table 2 below. These methods may be used to treat metastasized cancers, too, e.g., any of the cancers listed below, which has metastasized to another tissue.

TABLE 2

| Cancer | Antigens |
|---|---|
| Lung Cancer | VEGFR2, DLL3, DLL4, NECTIN1, NECTIN2, NECTIN3, |
| | NECTIN4, CD44v6, NKG2D, MAGE family (A-L types), WT1, |
| | KREMEN2, GRIN2D, CELSR3, KISS1R, CDH6, TREM1, MC1R, |
| | LIFR, PRR7, PTPRN, CAGNG7, TRPM1, Mesothelin, HER2, |
| | EphA2, PMSA, CD70, GD2, EGFR, EGFRviii, PSCA, MET, AXL, |
| | MCAM, Trop2, MUC1, ROR1, Claudin18.2, GPC3, CA9, EPCAM, |
| | FOLR1, L1CAM, CD133, CEACAM5, MUC16, PD-L1, ROR2 |
| Colorectal | VEGFR2, DLL3, DLL4, NECTIN1, NECTIN2, NECTIN3, |
| Cancer | NECTIN4, CD44v6, NKG2D, MAGE family (A-L types), WT1, |
| | KREMEN2, GRIN2D, CELSR3, KISS1R, CDH6, TREM1, MC1R, |
| | LIFR, PRR7, PTPRN, CAGNG7, TRPM1, EphA2, PMSA, CD70, |
| | ALPPL2, HER2, EGFR, PSCA, MET, AXL, MCAM, Trop2, MUC1, |
| | ROR1, Claudin18.2, GPC3, CA9, EPCAM, FOLR1, L1CAM, |
| | CD133, CEACAM5, PD-L1, ROR2 |
| Pancreatic | VEGFR2, DLL3, DLL4, NECTIN1, NECTIN2, NECTIN3, |
| Cancer | NECTIN4, CD44v6, NKG2D, MAGE family (A-L types), |
| | WT1, KREMEN2, GRIN2D, CELSR3, KISS1R, CDH6, TREM1, |
| | MC1R, LIFR, PRR7, PTPRN, CAGNG7, TRPM1Mesothelin, |
| | IL13Ra2, PMSA, ALPPL2, HER2, EphA2, EGFR, PSCA, MET, |
| | AXL, MCAM, Trop2, MUC1, ROR1, Claudin18.2, CA9, EPCAM, |
| | FOLR1, L1CAM, CD133, CEACAM5, MUC16, PD-L1, ROR2 |
| Prostate | ALK, VEGFR2, DLL3, DLL4, NECTIN1, NECTIN2, NECTIN3, |
| Cancer | NECTIN4, CD44v6, NKG2D, MAGE family (A-L types), WT1, |
| | KREMEN2, GRIN2D, CELSR3, KISS1R, CDH6, TREM1, MC1R, |
| | LIFR, PRR7, PTPRN, CAGNG7, TRPM1, EphA2, PMSA, HER2, |
| | EGFR, PSCA, AXL, MCAM, Trop2, MUC1, ROR1, EPCAM, |
| | CD133, PD-L1, ROR2 |
| Liver and | VEGFR2, DLL3, DLL4, NECTIN1, NECTIN2, NECTIN3, |
| biliary tract | NECTIN4, CD44v6, NKG2D, MAGE family (A-L types), WT1, |
| cancers | KREMEN2, GRIN2D, CELSR3, KISS1R, CDH6, TREM1, MC1R, |
| | LIFR, PRR7, PTPRN, CAGNG7, TRPM1Mesothelin, EGFR, |
| | EGFRviii, EphA2, MET, AXL, MCAM, MUC1, ROR1, |
| | Claudin18.2, GPC3, CA9, EPCAM, CD133, CEACAM5, |
| | PD-L1, ROR2 |
| Bladder | ALK, VEGFR2, DLL3, DLL4, NECTIN1, NECTIN2, NECTIN3, |
| Cancer | NECTIN4, CD44v6, NKG2D, MAGE family (A-L types), WT1, |
| | KREMEN2, GRIN2D, CELSR3, KISS1R, CDH6, TREM1, MC1R, |
| | LIFR, PRR7, PTPRN, CAGNG7, TRPM1, PMSA, , HER2, EGFR, |
| | EphA2, PSCA, MET, AXL, MCAM, Trop2, MUC1, ROR1, GPC3, |
| | CA9, EPCAM, L1CAM, CD133, CEACAM5, PD-L1, ROR2 |
| Esophageal | VEGFR2, DLL3, DLL4, NECTIN1, NECTIN2, NECTIN3, |
| Cancer | NECTIN4, CD44v6, NKG2D, MAGE family (A-L types), WT1, |
| | KREMEN2, GRIN2D, CELSR3, KISS1R, CDH6, TREM1, MC1R, |
| | LIFR, PRR7, PTPRN, CAGNG7, TRPM1, EphA2, AXL, MCAM, |
| | PD-L1, ROR2 |
| Ovarian | ALK, VEGFR2, DLL3, DLL4, NECTIN1, NECTIN2, NECTIN3, |
| Cancer | NECTIN4, CD44v6, NKG2D, MAGE family (A-L types), WT1, |
| | KREMEN2, GRIN2D, CELSR3, KISS1R, CDH6, TREM1, MC1R, |
| | LIFR, PRR7, PTPRN, CAGNG7, TRPM1, Mesothelin, HER2, |
| | Il13Ra2, EphA2, ALPPL2, GD2, EGFR, AXL, MCAM, Trop2, |
| | ROR1, GPC3, CA9, EPCAM, FOLR1, DLL3, DLL4, NECTIN1, |
| | NECTIN2, NECTIN3, NECTIN4, L1CAM, CD133, CEACAM5, |
| | MUC16, PD-L1, ROR2 |
| Kidney | VEGFR2, DLL3, DLL4, NECTIN1, NECTIN2, NECTIN3, |
| Cancer | NECTIN4, CD44v6, NKG2D, MAGE family (A-L types), WT1, |
| | KREMEN2, GRIN2D, CELSR3, KISS1R, CDH6, TREM1, MC1R, |
| | LIFR, PRR7, PTPRN, CAGNG7, TRPM1, EphA2, PMSA, CD70, |
| | GD2, HER2, EGFR, PSCA, MET, AXL, MCAM, Trop2, MUC1, |
| | ROR1, Claudin18.2, GPC3, CA9, EPCAM, FOLR1, L1CAM, |
| | CD133, PD-L1, ROR2 |
| Melanoma | MLANA (MART1), TYRP-1, ALK, VEGFR2, DLL3, DLL4, |
| | NECTIN1, NECTIN2, NECTIN3, NECTIN4, CD44v6, NKG2D, |
| | MAGE family (A-L types), WT1, KREMEN2, GRIN2D, CELSR3, |
| | KISS1R, CDH6, TREM1, MC1R, LIFR, PRR7, PTPRN, CAGNG7, |
| | TRPM1, IL13RA2, EphA2, CD70, MET, AXL, MCAM, Trop2, |
| | ROR1, L1CAM, PD-L1, ROR2 |
| Gastric/ | VEGFR2, DLL3, DLL4, NECTIN1, NECTIN2, NECTIN3, |
| Stomach | NECTIN4, CD44v6, NKG2D, MAGE family (A-L types), WT1, |
| Cancer | KREMEN2, GRIN2D, CELSR3, KISS1R, CDH6, TREM1, MC1R, |
| | LIFR, PRR7, PTPRN, CAGNG7, TRPM1, HER2, CD70, ALPPL2, |
| | EphA2, EGFR, PSCA, MET, AXL, MCAM, Trop2, MUC1, ROR1, |
| | Claudin18.2, GPC3, CA9, EPCAM, FOLR1, L1CAM, CD133, |
| | CEACAM5, MUC16, PD-L1, ROR2 |
| Breast | VEGFR2, DLL3, DLL4, NECTIN1, NECTIN2, NECTIN3, |
| Cancer | NECTIN4, CD44v6, NKG2D, MAGE family (A-L types), WT1, |
| | KREMEN2, GRIN2D, CELSR3, KISS1R, CDH6, TREM1, MC1R, |
| | LIFR, PRR7, PTPRN, CAGNG7, TRPM1, HER2, EphA2, PMSA, |
| | CD70, GD2, EGFR, PSCA, MET, AXL, MCAM, Trop2, MUC1, |
| | ROR1, Claudin18.2, GPC3, CA9, EPCAM, FOLR1, L1CAM, |
| | CD133, CEACAM5, PD-L1, ROR2 |
| Mesothelioma | VEGFR2, DLL3, DLL4, NECTIN1, NECTIN2, NECTIN3, |
| | NECTIN4, CD44v6, NKG2D, MAGE family (A-L types), WT1, |
| | KREMEN2, GRIN2D, CELSR3, KISS1R, CDH6, TREM1, MC1R, |
| | LIFR, PRR7, PTPRN, CAGNG7, TRPM1Mesothelin, ALPPL2, |
| | AXL, Claudin18.2, PD-L1, ROR2 |
| Uterine | VEGFR2, DLL3, DLL4, NECTIN1, NECTIN2, NECTIN3, |
| | NECTIN4, CD44v6, NKG2D, MAGE family (A-L types), WT1, |
| | KREMEN2, GRIN2D, CELSR3, KISS1R, CDH6, TREM1, MC1R, |
| | LIFR, PRR7, PTPRN, CAGNG7, TRPM1HER2, EphA2, Trop2, |
| | MUC1, ROR1, GPC3, CA9, EPCAM, FOLR1, L1CAM, CD133, |
| | CEACAM5, MUC16, PD-L1, ROR2 |
| testicular | VEGFR2, DLL3, DLL4, NECTIN1, NECTIN2, NECTIN3, |
| | NECTIN4, EphA2, PMSA, ALPPL2, AXL, MCAM, ROR1, GPC3, |
| | EPCAM, L1CAM, CD133, PD-L1, ROR2 |
| head and neck | VEGFR2, DLL3, DLL4, NECTIN1, NECTIN2, NECTIN3, |
| (including | NECTIN4, EphA2, HER2, EGFR, PSCA, MET, Trop2, MUC1, |
| thyroid) | ROR1, GPC3, CA9, EPCAM, L1CAM, CD133, CEACAM5, MUC16, |
| | PD-L1, ROR2 |

In some embodiments, an antigen may be selected from the following list: mesothelin, EGFRvIII, IL13RA2, EPHA2, PSMA (FOLH1), HER2, EGFR, PSCA, ALPPL2, GD2 (B4GALNT1), BCAN, MOG, CSPG5, CD70, MET, AXL, MCAM, DLL3, DLL4, nectin4, nectin2, nectin3, nectin1, and ALK.

In some embodiments, the subject has a cancer selected from the cancers listed in Table 2. In these embodiments, the cell administered to the subject has: (i) an anti-stromal marker BTTS; (ii) a nucleic acid encoding an immune receptor that is activated by binding to an antigen associated with the cancer in Table 2; and, optionally: (iii) a nucleic acid encoding a pro-inflammatory protein; In these embodiments, binding of the BTTS to the stromal marker on the surface of a stromal cell activates expression of the immune receptor of (ii) and, if present, the protein of (iii). The subject may have lung cancer, colorectal cancer, pancreatic cancer, prostate cancer, liver and/or biliary tract cancer, bladder cancer, esophageal cancer, ovarian cancer, kidney cancer, melanoma, gastric/stomach cancer, breast cancer, mesothelioma, uterine cancer, testicular cancer, or head and neck cancer (including thyroid), wherein the cancer is stromal marker⁺, for example.

Alternatively, the cell administered to the subject may have: i. an anti-stromal marker BTTS, as described above and ii. a nucleic acid encoding a pro-inflammatory protein. In these embodiments, binding of the BTTS to the stromal marker on the surface of a stromal cell activates expression of the pro-inflammatory protein of (ii). In these embodiments, the cell may further comprises an immune receptor that is activated by binding to GD2 or Her2, wherein expression of the immune receptor is constitutive in the cell.

As noted above, the immune receptor may be constitutively expressed (in which case its coding sequence will be operably linked to a constitutive promoter, i.e., a promoter that is always “on” in the cell), or induced by activation of the BTTS. In the latter case, the coding sequence for the pro-inflammatory protein and the coding sequence for the immune receptor may be both operably linked a single promoter (in which case the coding sequences may be separated by an IRES sequence, although other systems such as bidirectional promoters can be used), or they may be linked to different promoters, which may or may not have different sequences.

### Examples

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or see, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Recognition of FAP as a Priming Antigen for a Binding Triggered Transcriptional Switch.

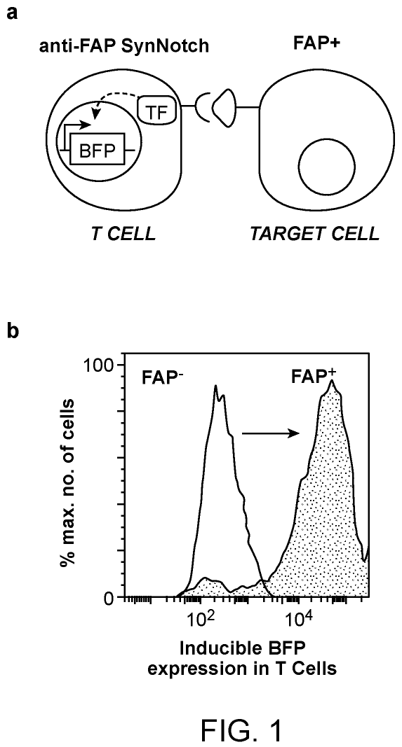

Human T cells were engineered to express a SynNotch receptor that drives production of a BFP reporter gene following receptor binding to target ligand, FAP. T cells were co-cultured with human pancreatic stellate cells (FAP+) or murine 3T3 fibroblasts (FAP negative) in-vitro for 24 hours and assayed for expression of BFP. BFP induction was only seen in T cells expressing the anti-FAP SynNotch driven BFP BTTS in the presence of FAP+priming cells. These results are show in FIG. 1.

### Recognition of FAP as a Priming Antigen for a Binding Triggered Transcriptional Switch to Drive CAR Expression.

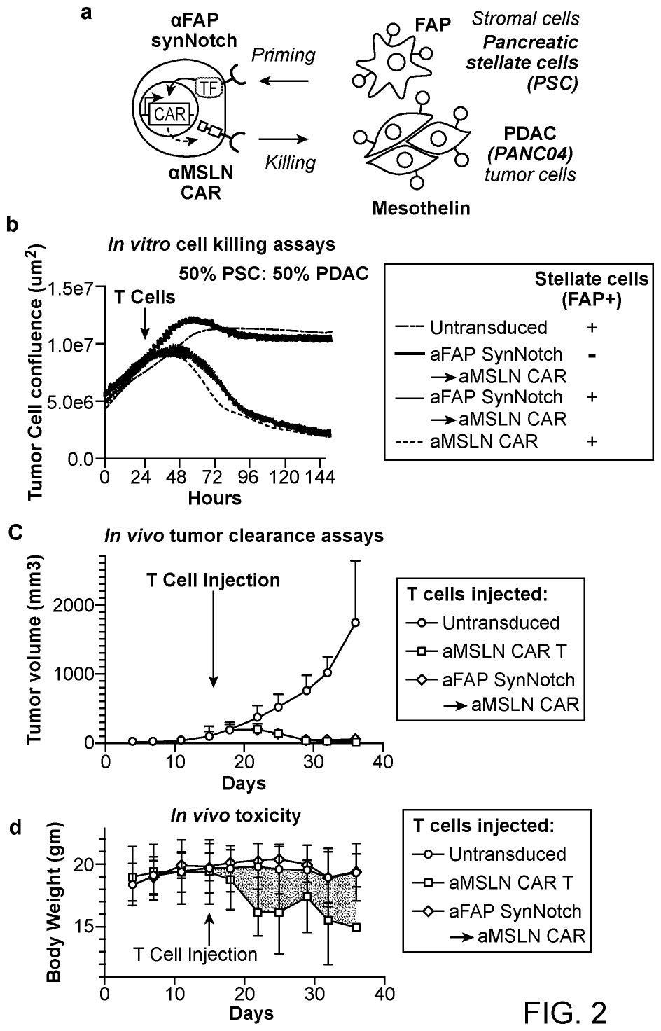

(a) FAP-and-Meso Human CD8+ CAR T cells were engineered to express a SynNotch receptor that drives production of an anti-mesothelin CAR following receptor binding to target ligand, FAP. T cells were co-cultured with human pancreatic stellate cells (FAP+) or murine 3T3 fibroblasts (FAP negative) as well as mesothelin expressing pancreatic cancer cells (PANC04) engineered to express GFP.
(b) T cell cytotoxicity was measured using an Incucycte live cell imaging system to assess GFP+ (pancreatic cancer) cell survival over time. Untransduced/unmodified human CD8+ T cells were used as a negative control and constitutively expressed anti-mesothelin CAR T cells were used as a positive control. T cell cytotoxicity against PANC04 cells was seen from FAP-and-Meso CAR T cells only with co-culture with FAP+ pancreatic stellate cells.
(c) PANC04 cells were implanted sub-cutaneously in the flank of NSG mice and allowed to engraft before treating mice with 6e6 of the indicated engineered T cell type (1:1 CD4:CD8). Both constitutive anti-mesothelin CAR T cells and FAP-and-Meso CAR T cells controlled tumor growth when compared to untransduced/unmodified T cells.
(d) T cell toxicity against mice was measured using an anti-mesothelin CAR that cross-reacts with mouse and human mesothelin. A reduction in body weight was noted when mice were treated with anti-mesothelin CAR T cells, but not with FAP-and-Meso CAR T cells.
   These results are shown in FIG. 2.

### Recognition of FAP as a Priming Antigen for a Binding Triggered Transcriptional Switch to Drive CAR and Cytokine Expression.

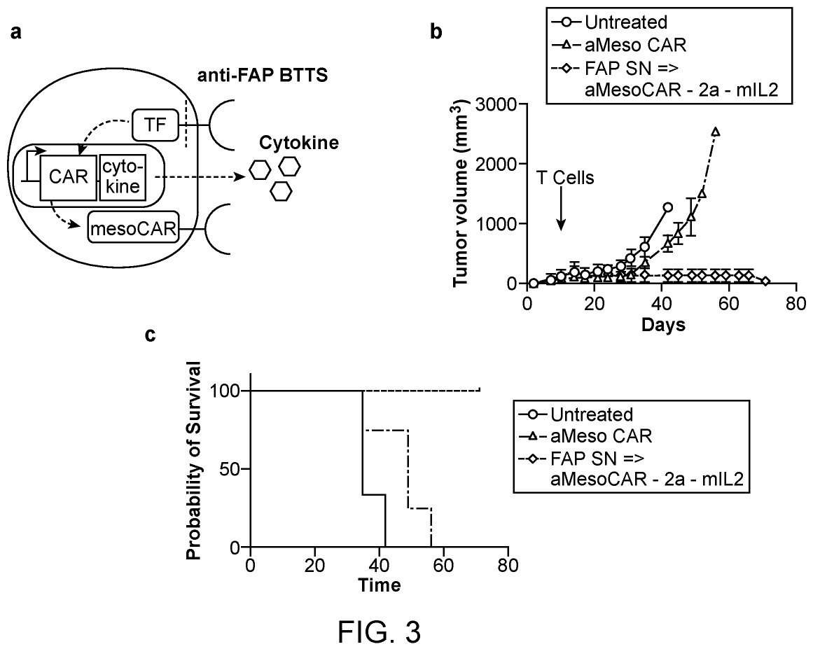

The following discussion is with reference to FIG. 3:
(a) FAP-and-Meso/IL2 Mouse T cells were engineered to express an anti-FAP BTTS (SynNotch) driving expression of both an anti-mesothelin CAR and IL-2 (linked by a p2a element).
(b) C57/B16 mice were implanted sub-cutaneously with the genetically engineered mouse model of pancreatic cancer, KPC. Therapeutic T cells were administered at a dose of 2e6 CD3+ cells sorted for receptor expression on tumor day +9. 150 mg/kg of Cytoxan was given as pre-conditioning on tumor day +8. Tumor growth was measured using serial caliper measurements of for each group. FAP-and-Meso/IL2 cells were compared to untransduced mouse T cells and constitutively expressed anti-mesothelin CAR T cells. Only transient tumor control was achieved by standard anti-mesothelin CAR T cells, while durable tumor control was achieved by FAP-and-Meso/IL2 CAR T cells.
(c) Kaplan-Meier survival curve for mice in FIG. 3b.
   Other Potential Anti-FAP BTTS Driven CAR Circuits Identified from an Analysis of RNA Expression in Tumor and Normal Tissue.

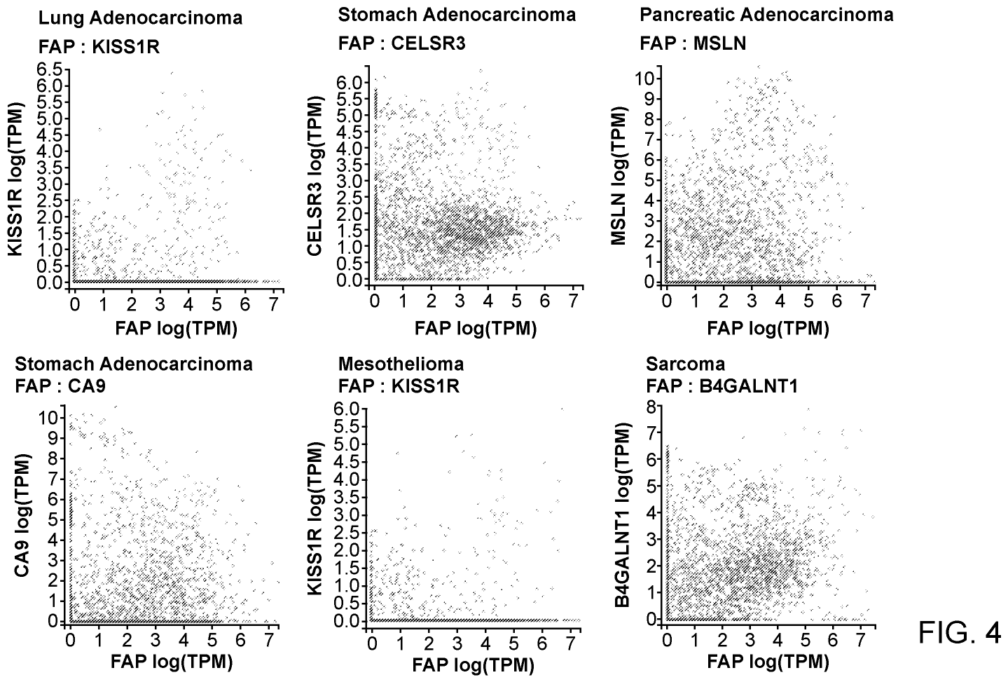

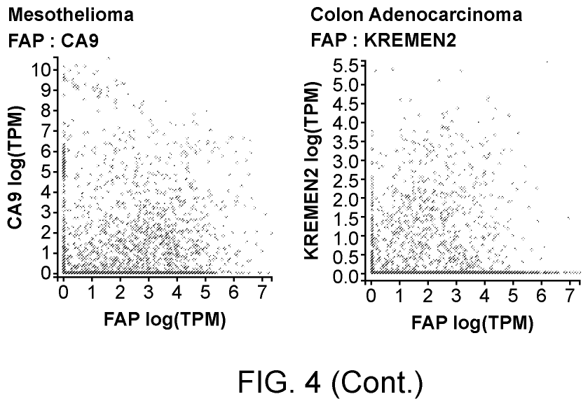

With reference to FIG. 4, tumor samples (red) from TCGA and normal tissue samples (grey) from GTEX were analyzed for expression of target antigens and plotted for indicated cancers. For each cancer listed a high performing antigen pair between the stromal antigen FAP and the indicated tumor associated antigen is depicted.

The following section provides the amino acid sequences of several polypeptide sequences that were used in the examples or that could be used herein. The Chothia CDRs are underlined in the antibody sequences.

### Sequences:

1) Anti-FAP F19 heavy chain:
(SEQ ID NO: 1)
MGWSWVFLFLLSGTAGVLSEVQLQQSGPELVKPGASVKMSCKTSRYTFTEYTIHWVR
QSHGKSLEWIGGINPNNGIPNYNQKFKGRATLTVGKSSSTAYMELRSLTSEDSAVYFCA
RRRIAYGYDEGHAMDYWGQGTSVTVSS
2) Anti-FAP F19 light chain:
(SEQ ID NO: 2)
MDSQAQVLMLLPLWVSGTCGDIVMSQSPSSLAVSVGEKVTMSCKSSQSLLYSRNQKN
YLAWFQQKPGQSPKLLIFWASTRESGVPDRFTGSGFGTDFNLTISSVQAEDLAVYDCQ
QYFSYPLTFGAGTKLELK
3) Anti-FAP Sibro Heavy Chain:
(SEQ ID NO: 3)
QVQLVQSGAEVKKPGASVKVSCKTSRYTFTEYTIHWVRQAPGQRLEWIGGINPNNGIP
NYNQKFKGRVTITVDTSASTAYMELSSLRSEDTAVYYCARRRIAYGYDEGHAMDYWG
QGTLVTVSS
4) Anti-FAP Sibro Light Chain:
(SEQ ID NO: 4)
DIVMTQSPDSLAVSLGERATINCKSSQSLLYSRNQKNYLAWYQQKPGQPPKLLIFWAS
TRESGVPDRFSGSGFGTDFTLTISSLQAEDVAVYYCQQYFSYPLTFGQGTKVEIK
5) Anti-FAP Mb34 Heavy Chain:
(SEQ ID NO: 5)
QVQLQQSGAEVKKPGSSVKVSCKASGGTFSTHTINWVRQAPGQGLEWMGGIAPMFGT
ANYAQKFQGRVTITADKSTSTAYMEMSSLRSDDTAVYYCARRRIAYGYDEGHAMDY
WGQGTLVTVSS
6) Anti-FAP Mb34 Light Chain:
(SEQ ID NO: 6)
DIQMTQSPSSLSASTGDRVTITCRASQDISSYLAWYQQAPGKAPHLLMSGATTLQTGVP
SRFSGSGSGTDFTLTISSLQAEDVAVYYCQQYYRTPFTFGQGTKLEIK
7) Anti-FAP 28H1 Heavy chain:
(SEQ ID NO: 7)
EIVLTQSPGTLSLSPGERATLSCRASQSVSRSYLAWYQQKPGQAPRLLIIGASTRATGIP
DRFSGSGSGTDFTLTISRLEPEDFAVYYCQQGQVIPPTFGQGTKVEIK
8) Anti-FAP 28H1 Light Chain:
(SEQ ID NO: 8)
EVQLLESGGGLVQPGGSLRLSCAASGFTFSSHAMSWVRQAPGKGLEWVSAIWASGEQ
YYADSVKGRFTISRDNSKNTLYLQMNSLRAEDTAVYYCAKGWLGNFDYWGQGTLVT
VSS
9) Anti-Mesothelin SS1 Heavy Chain:
(SEQ ID NO: 9)
QVQLQQSGPELEKPGASVKISCKASGYSFTGYTMNWVKQSHGKSLEWIGLITPYNGAS
SYNQKFRGKATLTVDKSSSTAYMDLLSLTSEDSAVYFCARGGYDGRGFDYWGQGTTV
TVSS
10) Anti-Mesothelin SS1 Light Chain:
(SEQ ID NO: 10)
DIELTQSPAIMSASPGEKVTMTCSASSSVSYMHWYQQKSGTSPKRWIYDTSKLASGVPG
RFSGSGSGNSYSLTISSVEAEDDATYYCQQWSKHPLTYGAGTKLEIKAS
11) Anti-Mesothelin g3a nanobody:
(SEQ ID NO: 11)
QVQLVQSGGGLVHPGGSLRLSCAASGIDLSLYRMRWYRQAPGKERDLVALITDDGTS
YYEDSVKGRFTITRDNPSNKVFLQMNSLKPEDTAVYYCNAETPLSPVNYWGQGTQVT
VS
12) Anti-Mesothelin m912 Heavy Chain:
(SEQ ID NO: 12)
QVQLQESGPGLVKPSETLSLTCTVSGGSVSSGSYYWSWIRQPPGKGLEWIGYIYYSGST
NYNPSLKSRVTISVDTSKNQFSLKLSSVTAADTAVYYCAREGKNGAFDIWGQGTMVTV
SS
13) Anti-Mesothelin m912 Light Chain:
(SEQ ID NO: 13)
RHQMTQSPSSLSASVGDRVTITCRASQSISSYLNWYQQKPGKAPKLLIYAASSLQSGVP
SRFSGSGSGTDFTLTISSLQPEDFATYYCQQSYSTPLTFGGGTKVEIK
14) Anti-Mesothelin m5 Heavy Chain:
(SEQ ID NO: 14)
QVQLVQSGAEVEKPGASVKVSCKASGYTFTDYYMHWVRQAPGQGLEWMGWINPNS
GGTNYAQKFQGRVTMTRDTSISTAYMELSRLRSDDTAVYYCASGWDFDYWGQGTLV
TVSS
15) Anti-Mesothelin m5 Light Chain:
(SEQ ID NO: 15)
DIVMTQSPSSLSASVGDRVTITCRASQSIRYYLSWYQQKPGKAPKLLIYTASILQNGVPS
RFSGSGSGTDFTLTISSLQPEDFATYYCLQTYTTPDFGPGTKVEIK
16) Cytokine mouse IL-2:
(SEQ ID NO: 16)
MYSMQLASCVTLTLVLLVNSAPTSSSTSSSTAEAQQQQQQQQQQQQHLEQLLMDLQEL
LSRMENYRNLKLPRMLTFKFYLPKQATELKDLQCLEDELGPLRHVLDLTQSKSFQLEDA
ENFISNIRVTVVKLKGSDNTFECQFDDESATVVDFLRRWIAFCQSIISTSPQ
17) Cytokine human IL-2:
(SEQ ID NO: 17)
MYRMQLLSCIALSLALVTNSAPTSSSTKKTQLQLEHLLLDLQMILNGINNYKNPKLTRM
LTFKFYMPKKATELKHLQCLEEELKPLEEVLNLAQSKNFHLRPRDLISNINVIVLELKGSE
TTFMCEYADETATIVEFLNRWITFCQSIISTLT
18) Cytokine mouse IL-7:
(SEQ ID NO: 18)
MFHVSFRYIFGIPPLILVLLPVTSSECHIKDKEGKAYESVLMISIDELDKMTGTDSNCPNN
EPNFFRKHVCDDTKEAAFLNRAARKLKQFLKMNISEEFNVHLLTVSQGTQTLVNCTSKE
EKNVKEQKKNDACFLKRLLREIKTCWNKILKGSI
19) Cytokine human IL-7:
(SEQ ID NO: 19)
MFHVSFRYIFGLPPLILVLLPVASSDCDIEGKDGKQYESVLMVSIDQLLDSMKEIGSNCL
NNEFNFFKRHICDANKEGMFLFRAARKLRQFLKMNSTGDFDLHLLKVSEGTTILLNCTG
QVKGRKPAALGEAQPTKSLEENKSLKEQKKLNDLCFLKRLLQEIKTCWNKILMGTKEH
20) Cytokine mouse IL-15:
(SEQ ID NO: 20)
MKILKPYMRNTSISCYLCFLLNSHFLTEAGIHVFILGCVSVGLPKTEANWIDVRYDLEKIE
SLIQSIHIDTTLYTDSDFHPSCKVTAMNCFLLELQVILHEYSNMTLNETVRNVLYLANSTL
SSNKNVAESGCKECEELEEKTFTEFLQSFIRIVQMFINTS
21) Cytokine human IL-15:
(SEQ ID NO: 21)
MRISKPHLRSISIQCYLCLLLNSHFLTEAGIHVFILGCFSAGLPKTEANWVNVISDLKKIED
LIQSMHIDATLYTESDVHPSCKVTAMKCFLLELQVISLESGDASIHDTVENLIILANNSLSS
NGNVTESGCKECEELEEKNIKEFLQSFVHIVQMFINTS
22) Cytokine mouse IL-21:
(SEQ ID NO: 22)
MERTLVCLVVIFLGTVAHKSSPQGPDRLLIRLRHLIDIVEQLKIYENDLDPELLSAPQDVK
GHCEHAAFACFQKAKLKPSNPGNNKTFIIDLVAQLRRRLPARRGGKKQKHIAKCPSCDS
YEKRTPKEFLERLKWLLQKMIHQHLS
23) Cytokine human Il-21:
(SEQ ID NO: 23)
MRSSPGNMERIVICLMVIFLGTLVHKSSSQGQDRHMIRMRQLIDIVDQLKNYVNDLVPE
FLPAPEDVETNCEWSAFSCFQKAQLKSANTGNNERIINVSIKKLKRKPPSTNAGRRQKHR
LTCPSCDSYEKKPPKEFLERFKSLLQKMIHQHLSSRTHGSEDS
24) Cytokine DR-18:
(SEQ ID NO: 24)
MAAMSEDSCVNFKEMMFIDNTLYFIPEENGDLESDHFGRLHCTTAVIRNINDQVLFVDK
RQPVFEDMTDIDQSASEPQTRLIIYAYGDSRARGKAVTLSVKDSKMSTLSCKNKIISFEE
MDPPENIDDIQSDLIFFQKRVPGHNKMEFESSLYEGHFLACQKEDDAFKLILKKKDENG
DKSVMFTLTNLHQS
25) Cytokine mouse CD40L:
(SEQ ID NO: 25)
IETYSQPSPRSVATGLPASMKIFMYLLTVFLITQMIGSVLFAVYLHRRLDKVEEEVNLHE
DFVFIKKLKRCNKGEGSLSLLNCEEMRRQFEDLVKDITLNKEEKKENSFEMQRGDEDPQ
IAAHVVSEANSNAASVLQWAKKGYYTMKSNLVMLENGKQLTVKREGLYYVYTQVTF
CSNREPSSQRPFIVGLWLKPSSGSERILLKAANTHSSSQLCEQQSVHLGGVFELQAGASV
FVNVTEASQVIHRVGFSSFGLLKL
26) Cytokine human CD40L:
(SEQ ID NO: 26)
MIETYNQTSPRSAATGLPISMKIFMYLLTVFLITQMIGSALFAVYLHRRLDKIEDERNLHE
DFVFMKTIQRCNTGERSLSLLNCEEIKSQFEGFVKDIMLNKEETKKENSFEMQKGDQNP
QIAAHVISEASSKTTSVLQWAEKGYYTMSNNLVTLENGKQLTVKRQGLYYIYAQVTFC
SNREASSQAPFIASLCLKSPGRFERILLRAANTHSSAKPCGQQSIHLGGVFELQPGASVFV
NVTDPSQVSHGTGFTSFGLLKL
27) Cytokine mouse CCL21:
(SEQ ID NO: 27)
MAQMMTLSLLSLVLALCIPWTQGSDGGGQDCCLKYSQKKIPYSIVRGYRKQEPSLGCPI
PAILFSPRKHSKPELCANPEEGWVQNLMRRLDQPPAPGKQSPGCRKNRGTSKSGKKGK
GSKGCKRTEQTQPSRG
28) Cytokine human CCL21:
(SEQ ID NO: 28)
MAQSLALSLLILVLAFGIPRTQGSDGGAQDCCLKYSQRKIPAKVVRSYRKQEPSLGCSIP
AILFLPRKRSQAELCADPKELWVQQLMQHLDKTPSPQKPAQGCRKDRGASKTGKKGK
GSKGCKRTERSQTPKGP
29) Cytokine mouse IL-12:
(SEQ ID NO: 29)
MCPQKLTISWFAIVLLVSPLMAMWELEKDVYVVEVDWTPDAPGETVNLTCDTPEEDDI
TWTSDQRHGVIGSGKTLTITVKEFLDAGQYTCHKGGETLSHSHLLLHKKENGIWSTEIL
KNFKNKTFLKCEAPNYSGRFTCSWLVQRNMDLKFNIKSSSSSPDSRAVTCGMASLSAEK
VTLDQRDYEKYSVSCQEDVTCPTAEETLPIELALEARQQNKYENYSTSFFIRDIIKPDPPK
NLQMKPLKNSQVEVSWEYPDSWSTPHSYFSLKFFVRIQRKKEKMKETEEGCNQKGAFL
VEKTSTEVQCKGGNVCVQAQDRYYNSSCSKWACVPCRVRSGGGGSGGGGSGGGGSR
VIPVSGPARCLSQSRNLLKTTDDMVKTAREKLKHYSCTAEDIDHEDITRDQTSTLKTCLP
LELHKNESCLATRETSSTTRGSCLPPQKTSLMMTLCLGSIYEDLKMYQTEFQAINAALQN
HNHQQIILDKGMLVAIDELMQSLNHNGETLRQKPPVGEADPYRVKMKLCILLHAFSTRV
VTINRVMGYLSSA
30) Cytokine human IL-12:
(SEQ ID NO: 30)
MCHQQLVISWFSLVFLASPLVAIWELKKDVYVVELDWYPDAPGEMVVLTCDTPEEDGI
TWTLDQSSEVLGSGKTLTIQVKEFGDAGQYTCHKGGEVLSHSLLLLHKKEDGIWSTDIL
KDQKEPKNKTFLRCEAKNYSGRFTCWWLTTISTDLTFSVKSSRGSSDPQGVTCGAATLS
AERVRGDNKEYEYSVECQEDSACPAAEESLPIEVMVDAVHKLKYENYTSSFFIRDIIKPD
PPKNLQLKPLKNSRQVEVSWEYPDTWSTPHSYFSLTFCVQVQGKSKREKKDRVFTDKT
SATVICRKNASISVRAQDRYYSSSWSEWASVPCSGGGGSGGGGSGGGGSRNLPVATPDP
GMFPCLHHSQNLLRAVSNMLQKARQTLEFYPCTSEEIDHEDITKDKTSTVEACLPLELTK
NESCLNSRETSFITNGSCLASRKTSFMMALCLSSIYEDLKMYQVEFKTMNAKLLMDPKR
QIFLDQNMLAVIDELMQALNFNSETVPQKSSLEEPDFYKTKIKLCILLHAFRIRAVTIDRV
MSYLNAS

While the foregoing reduction to practice has been implemented using FAP, the principle can be applied to other stromal markers such as PDPN (Podoplanin), CDH11 (Cadherin 11), PDGFR (Platelet-derived growth factor) or LRRC15 (Leucine Rich Repeat Containing protein 15).

### Targeting Lung Cancer In Vivo Using a FAP-EGFR Circuit

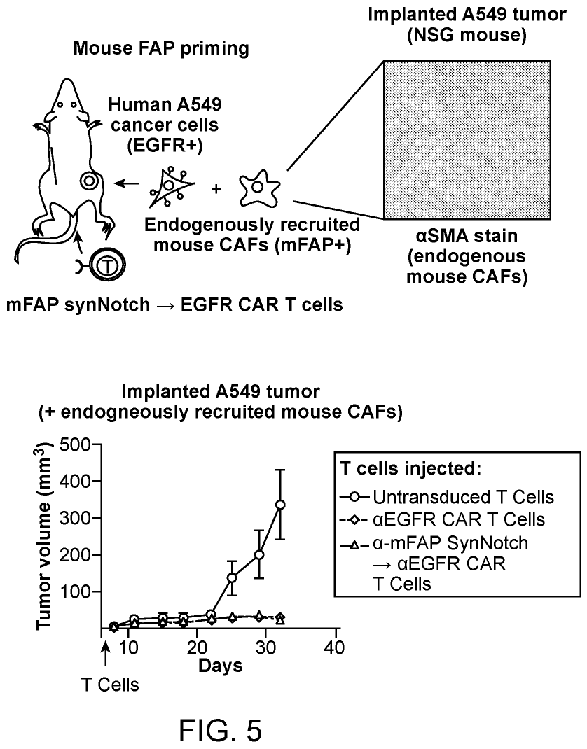

In this example, stromal priming was used to localize CAR T cell activity to a tumor micro-environment in a model of non-small cell lung cancer. Here the human lung cancer xenograft cell line A549 was implanted in immunodeficient NSG mice and treated with therapeutic human T cells. 2.5e6 tumor cells were implanted sub-cutaneously with T cell treatment given on tumor day 17. Therapeutic T cells were either control untransduced CD4/CD8 human T cells (blue), standard constitutively active anti-EGFR CAR CD4/CD8 human T cells (brown) or anti-FAP SynNotch=>anti-EGFR CAR CD4/CD8 human T cells (red). Excellent tumor control was seen with both the constitutive and the FAP synNotch inducible anti-EGFR CAR T cell design. n=5 mice per group, mean+/−s.d. shown. These results are shown in FIG. 5.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. An engineered immune cell comprising a molecular circuit comprising the following components:
(a) an anti-stromal marker binding-triggered transcriptional switch (BTTS) and one or both of:
(b) a nucleic acid encoding a pro-inflammatory protein, and
(c) a nucleic acid encoding an immune receptor that is activated by binding to an antigen listed in Table 1 on a cancer cell;
wherein the stromal marker is: (i) FAP (Fibroblast Activation Protein Alpha), (ii) PDPN (Podoplanin), (iii) CDH11 (Cadherin 11), (iv) PDGFR (Platelet-derived growth factor) or (v) LRRC15 (Leucine Rich Repeat Containing protein 15), and
wherein binding of the BTTS to the stromal on the surface of a stromal cell activates expression of the pro-inflammatory protein of (b) and/or the immune receptor of (c).

2. The cell of claim 1, wherein the circuit comprises components (a), (b) and (c).

3. The cell of claim 1, wherein the circuit comprises components (a) and (b).

4. The cell of claim 1, wherein the circuit comprises components (a) and (c).

5. An engineered immune cell comprising the following components:
(a) an anti-stromal marker BTTS;
(b) a nucleic acid encoding a pro-inflammatory protein,
wherein the stromal marker is: (i) FAP (Fibroblast Activation Protein Alpha), (ii) PDPN (Podoplanin), (iii) CDH11 (Cadherin 11), (iv) PDGFR (Platelet-derived growth factor) or (v) LRRC15 (Leucine Rich Repeat Containing protein 15), and
wherein binding of the BTTS to the stromal marker on the surface of a stromal cell activates expression of the pro-inflammatory protein of (b); and
(c) an immune receptor that is activated by binding to an antigen listed in Table 1 on a cancer cell, wherein expression of the immune receptor is constitutive in the cell.

6. The cell of claim 1, wherein the pro-inflammatory protein encoded by the nucleic acid of (b) is IL-2, CCL-21, IL-12, IL-7, IL-15, IL-21, or a non-natural variant thereof that has pro-inflammatory activity.

7. The cell of claim 1, wherein the BTTS comprises an extracellular domain that comprises a scFv or nanobody that binds to the stromal marker.

8. The cell of claim 1, wherein the BTTS is a SynNotch receptor, an A2 receptor, a MESA, or another receptor that undergoes binding induced proteolytic cleavage.

9. The cell of claim 1, wherein the BTTS comprises:
i. an extracellular binding domain that binds to the stromal marker,
ii. a force sensing region,
iii. a transmembrane domain,
iv. one or more force-dependent cleavage sites that are cleaved when the force sensing region is activated, and
v. an intracellular domain comprising a transcriptional activator, where binding of the extracellular binding domain to the stromal marker on the surface of another cell induces proteolytic cleavage of the one or more force-dependent cleavage sites to release the transcriptional activator, and
wherein the transcriptional activator induces expression of the pro-inflammatory protein and/or immune receptor.

10. The cell of claim 1, wherein the cell is a myeloid or lymphoid cell.

11. The cell of claim 1, wherein the cell a T lymphocyte, a B lymphocyte, a macrophage or a Natural Killer cell.

12. The cell of claim 1, wherein the immune receptor is a chimeric antigen receptor (TCR) or engineered T cell receptor (TCR).

13. The cell of claim 1, wherein the pro-inflammatory protein is a pro-inflammatory cytokine.

14. The cell of claim 1, wherein the BTTS comprises one or more protease cleavage sites and an intracellular domain comprising a transcriptional activator, wherein binding of the BTTS to the tissue- or a cancer-associated antigen on another cell causes the BTTS to be cleaved at the protease cleavage site, thereby releasing the transcriptional activator, and wherein the released transcriptional activator induces expression of the pro-inflammatory protein.

15. The cell of claim 14, wherein the one or more protease cleavage sites comprises a cleavage site for a γ-secretase

16. A method of treating a subject for a cancer that is positive for stromal marker, comprising:
administering to the subject a cell of claim 1.

17. The method of claim 14, wherein:
(a) the subject has a cancer selected from the cancers listed in Table 2 and the cell administered to the subject has:
(i) an anti-stromal marker BTTS, wherein the stromal marker is: (i) FAP (Fibroblast Activation Protein Alpha), (ii) PDPN (Podoplanin), (iii) CDH11 (Cadherin 11), (iv) PDGFR (Platelet-derived growth factor) or (v) LRRC15 (Leucine Rich Repeat Containing protein 15),
(ii) a nucleic acid encoding an immune receptor that is activated by binding to an antigen associated with the cancer in Table 2; and, optionally:
(iii) a nucleic acid encoding a pro-inflammatory protein;
wherein binding of the BTTS to the stromal marker on the surface of a stromal cell activates expression of the immune receptor of (ii) and, if present, the protein of (iii).

18. The method of claim 17, wherein the subject has lung cancer, colorectal cancer, pancreatic cancer, prostate cancer, liver and/or biliary tract cancer, bladder cancer, esophageal cancer, ovarian cancer, kidney cancer, melanoma, gastric/stomach cancer, breast cancer, mesothelioma, uterine cancer, testicular cancer, or head and neck cancer (including thyroid).


SEQUENCE LISTING (36 sequences)

SEQ ID NO: 1 - protein, 143 aa, synthetic construct
MGWSWVFLFLLSGTAGVLSEVQLQQSGPELVKPGASVKMSCKTSRYTFTEYTIHWVRQSHGKSLEWIGGINPNNGIPNYNQKFKGRATLTVGKSSSTAYMELRSLTSEDSAVYFCARRRIAYGYDEGHAMDYWGQGTSVTVSS

SEQ ID NO: 2 - protein, 133 aa, synthetic construct
MDSQAQVLMLLPLWVSGTCGDIVMSQSPSSLAVSVGEKVTMSCKSSQSLLYSRNQKNYLAWFQQKPGQSPKLLIFWASTRESGVPDRFTGSGFGTDFNLTISSVQAEDLAVYDCQQYFSYPLTFGAGTKLELK

SEQ ID NO: 3 - protein, 124 aa, synthetic construct
QVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSS

SEQ ID NO: 4 - protein, 113 aa, synthetic construct
DIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIK

SEQ ID NO: 5 - protein, 124 aa, synthetic construct
QVQLQQSGAEVKKPGSSVKVSCKASGGTFSTHTINWVRQAPGQGLEWMGGIAPMFGTANYAQKFQGRVTITADKSTSTAYMEMSSLRSDDTAVYYCARRRIAYGYDEGHAMDYWGQGTLVTVSS

SEQ ID NO: 6 - protein, 107 aa, synthetic construct
DIQMTQSPSSLSASTGDRVTITCRASQDISSYLAWYQQAPGKAPHLLMSGATTLQTGVPSRFSGSGSGTDFTLTISSLQAEDVAVYYCQQYYRTPFTFGQGTKLEIK

SEQ ID NO: 7 - protein, 108 aa, synthetic construct
EIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIK

SEQ ID NO: 8 - protein, 116 aa, synthetic construct
EVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSS

SEQ ID NO: 9 - protein, 119 aa, synthetic construct
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSS

SEQ ID NO: 10 - protein, 108 aa, synthetic construct
DIELTQSPAIMSASPGEKVTMTCSASSSVSYMHWYQQKSGTSPKRWIYDTSKLASGVPGRFSGSGSGNSYSLTISSVEAEDDATYYCQQWSKHPLTYGAGTKLEIKAS

SEQ ID NO: 11 - protein, 116 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVS

SEQ ID NO: 12 - protein, 119 aa, synthetic construct
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSS

SEQ ID NO: 13 - protein, 107 aa, synthetic construct
RHQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 14 - protein, 115 aa, synthetic construct
QVQLVQSGAEVEKPGASVKVSCKASGYTFTDYYMHWVRQAPGQGLEWMGWINPNSGGTNYAQKFQGRVTMTRDTSISTAYMELSRLRSDDTAVYYCASGWDFDYWGQGTLVTVSS

SEQ ID NO: 15 - protein, 106 aa, synthetic construct
DIVMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIK

SEQ ID NO: 16 - protein, 169 aa, synthetic construct
MYSMQLASCVTLTLVLLVNSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 17 - protein, 153 aa, synthetic construct
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 18 - protein, 154 aa, synthetic construct
MFHVSFRYIFGIPPLILVLLPVTSSECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 19 - protein, 177 aa, synthetic construct
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 20 - protein, 162 aa, synthetic construct
MKILKPYMRNTSISCYLCFLLNSHFLTEAGIHVFILGCVSVGLPKTEANWIDVRYDLEKIESLIQSIHIDTTLYTDSDFHPSCKVTAMNCFLLELQVILHEYSNMTLNETVRNVLYLANSTLSSNKNVAESGCKECEELEEKTFTEFLQSFIRIVQMFINTS

SEQ ID NO: 21 - protein, 162 aa, synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 22 - protein, 146 aa, synthetic construct [sequence omitted]

SEQ ID NO: 23 - protein, 162 aa, synthetic construct
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 24 - protein, 192 aa, synthetic construct
MAAMSEDSCVNFKEMMFIDNTLYFIPEENGDLESDHFGRLHCTTAVIRNINDQVLFVDKRQPVFEDMTDIDQSASEPQTRLIIYAYGDSRARGKAVTLSVKDSKMSTLSCKNKIISFEEMDPPENIDDIQSDLIFFQKRVPGHNKMEFESSLYEGHFLACQKEDDAFKLILKKKDENGDKSVMFTLTNLHQS

SEQ ID NO: 25 - protein, 259 aa, synthetic construct
IETYSQPSPRSVATGLPASMKIFMYLLTVFLITQMIGSVLFAVYLHRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKL

SEQ ID NO: 26 - protein, 261 aa, synthetic construct
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 27 - protein, 133 aa, synthetic construct
MAQMMTLSLLSLVLALCIPWTQGSDGGGQDCCLKYSQKKIPYSIVRGYRKQEPSLGCPIPAILFSPRKHSKPELCANPEEGWVQNLMRRLDQPPAPGKQSPGCRKNRGTSKSGKKGKGSKGCKRTEQTQPSRG

SEQ ID NO: 28 - protein, 134 aa, synthetic construct
MAQSLALSLLILVLAFGIPRTQGSDGGAQDCCLKYSQRKIPAKVVRSYRKQEPSLGCSIPAILFLPRKRSQAELCADPKELWVQQLMQHLDKTPSPQKPAQGCRKDRGASKTGKKGKGSKGCKRTERSQTPKGP

SEQ ID NO: 29 - protein, 543 aa, synthetic construct
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 30 - protein, 540 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 31 - protein, 10 aa, synthetic construct
GRRRRELDPM

SEQ ID NO: 32 - protein, 9 aa, synthetic construct
RQRRELDPM

SEQ ID NO: 33 - protein, 8 aa, synthetic construct
KIEAVKSE

SEQ ID NO: 34 - protein, 8 aa, synthetic construct
KIEAVQSE

SEQ ID NO: 35 - protein, 8 aa, synthetic construct
VGCGVLLS

SEQ ID NO: 36 - protein, 7 aa, synthetic construct
GCGVLLS